(19) United States
(12) Patent Application Publication
(10) Pub. No.: US 2025/0263436 A1
(43) Pub. Date: Aug. 21, 2025

(54) BROAD-SPECTRUM ANTIVIRAL DRUG FOR ENTEROVIRUS, AND APPLICATION

(71) Applicant: WUHAN INSTITUTE OF VIROLOGY, CHINESE ACADEMY OF SCIENCES, Wuhan (CN)

(72) Inventors: Xi ZHOU, Wuhan (CN); Yuan FANG, Wuhan (CN); Yang QIU, Wuhan (CN); Di WU, Wuhan (CN); Muhan HUANG, Wuhan (CN); Ting SHU, Wuhan (CN)

(73) Assignee: WUHAN INSTITUTE OF VIROLOGY, CHINESE ACADEMY OF SCIENCES, Wuhan (CN)

(21) Appl. No.: 18/018,518

(22) PCT Filed: Mar. 19, 2021
(86) PCT No.: PCT/CN2021/081723

§ 371 Date: Jan. 27, 2023

(30) Foreign Application Priority Data
Jul. 28, 2020 (CN) 202010735992.8

(51) Int. Cl. C07K 7/08; A61K 38/10; A61P 31/14; C07K 19/00

(52) U.S. Cl. CPC C07K 7/08; A61K 38/10; A61P 31/14; C07K 19/00

(57) ABSTRACT

One of the core sequences of a polypeptide inhibitor provided by the present invention is as shown in SEQ ID NO.1, and the sequence of a polypeptide comprising a cell-penetrating peptide is as shown in SEQ ID NO.2. The polypeptide provided by the present invention uses enterovirus 2C protein multimerization as a target. Compared with other inhibitors targeting an enterovirus 2C protein, the present invention has high inhibition efficiency, good safety, and provides a new policy for enterovirus prevention and control.

### CROSS REFERENCE TO RELATED APPLICATIONS

This application is a national phase application under 35 U.S.C. § 371 of International Application No. PCT/CN2021/081723, filed Mar. 19, 2021, which claims the priority of Chinese Patent Application No. 202010735992.8, filed Jul. 28, 2020, each of which are hereby incorporated by reference in their entirety.

### REFERENCE TO A SEQUENCE LISTING

Pursuant to 37 C.F.R. 1.821 (c), a sequence listing is submitted herewith as an ASCII compliant text file named “UNITP0094US_Corrected_Sequence_Listing”, created on Sep. 1, 2023, and having a size of ˜10 kilobytes. The content of the aforementioned file is hereby incorporated by reference in its entirety.

### FIELD

The present invention relates to the technical field of biomedicine, and in particular to a broad-spectrum antiviral drug for enterovirus and an application thereof.

### BACKGROUND

Enterovirus, as a positive-sense single-stranded RNA virus, belongs to the Enterovirus genus of the Picornaviridae family, and mainly includes human enterovirus (EV), coxsackie A virus (CVA), coxsackie B virus (CVB), echovirus, rhinovirus, poliovirus, etc. Enterovirus infections are widely distributed all over the world, and exhibit complex and diverse clinical manifestations, ranging from mild low-grade fever, fatigue and respiratory diseases, to herpetic angina, hand-foot-mouth disease, severe aseptic meningitis, myocarditis, encephalitis, poliomyelitis and the like. At present, there are no specific drugs for effectively treating or preventing against enterovirus infections.

Herpetic angina is mainly caused by coxsackie A virus type 2 (CVA2), CVA4, CVA6, CVA9, CVA16, CVA22, coxsackie B virus type 1 (CVB1), CVB2, CVB3, CVB4 or CVB5. Herpetic angina often presents with acute fever of mostly low or moderate degree and occasionally as high as 40° C. or above, and even causes convulsion. The course of the fever is about 2-4 days. Older children may complain of sore throat which can affect swallowing. Infants and young children present with salivation, refusal to eat, and restlessness, sometimes accompanied by headache, stomachache or myalgia. About 25% of children under 5 years old may be accompanied by vomiting. Typical symptoms appear in the pharynx, and manifest as hyperemia in the pharynx, and several (as few as 1-2, or as many as 10) small (1-2 mm in diameter) grey-white herpes surrounded by redness in the oral mucosa within 2 days of onset. After 2-3 days, the redness intensifies and expands, and the herpes break out to form yellow ulcers. Such mucosal herpes commonly appear in the tonsil anterior pillar, and can also appear in the soft palate, uvula and tonsils, but do not involve the gums and buccal mucosa. The course of the disease is generally 4-6 days, and occasionally extends to 2 weeks.

Hand-foot-mouth disease is mainly caused by enterovirus 71 (EV71), CVA6, CVA8, CVA10, CVA16, CVB3 and CVB5, and has common clinical manifestations of acute fever, mouth pain, anorexia, and scattered herpes or ulcers in the oral mucosa, which mostly appear in the tongue, buccal mucosa, and hard palate, and can also appear in the soft palate, gums, tonsils, and pharynx. Maculopapular rashes appear on hands, feet, buttocks, arms, and legs, and then turn into herpes, which may be surrounded by inflammatory redness and have little liquid inside. There are more rashes on hands and feet, both on the dorsum and the vola from a few to dozens. After the rashes subside, no trace and no pigmentation are left. Some children with hand-foot-mouth disease present with herpetic angina as the first symptom, and then red rashes on palms, soles, and buttocks. When the disease develops rapidly, a small number of children can develop from hand-foot-and-mouth disease to severe aseptic meningitis and encephalitis, manifested as fever, headache, nausea, vomiting, and then meningeal irritation, as well as great fluctuation in body temperature, with low-grade fever in the most case and sometimes with fever up to 40° C. or above, often bimodal fever in the course of the disease. Other symptoms include such as sore throat, muscle aches, skin rash, photophobia, diarrhea, swollen lymph nodes, and sometimes mild paralysis.

Myocarditis is mainly caused by CVB1-61 and Echovirus. The clinical manifestations of patients with viral myocarditis depend on the extent and location of the lesion. Mild cases can be asymptomatic, while severe cases can present with heart failure, cardiogenic shock and sudden death. Patients often have a history of infection in upper respiratory tract or intestines 1-3 weeks before the onset, manifested as symptoms such as fever, body aches, sore throat, fatigue, nausea, vomiting, and diarrhea, followed by palpitations, chest tightness, chest pain or precordial dull pain, dizziness, dyspnea, edema, and even Adams-Stokes syndrome. An extremely small number of patients develop heart failure or cardiogenic shock.

Enterovirus is a positive-sense single-stranded RNA virus with a genome of about 7.5 kb containing a large ORF that encodes a polyprotein. The polyprotein is further hydrolyzed into 4 structural proteins (VP1-VP4) and 7 non-structural proteins (2A-2C and 3A-3D). Protein 2C is a very conservative non-structural protein in enterovirus (including EV71, CVA, CVB, echovirus, etc.), and exists in the form of homopolymer. Enterovirus protein 2C has an activity of RNA helicase, and is a classical superfamily 3 (SF3) helicase. A large number of studies on EV71 and PV have proved that the helicase activity of 2C is necessary for the replication and proliferation of the virus, and the multimerization of protein 2C is crucial for its helicase function. Therefore, the present invention designs a polypeptide drug targeting the multimerization domain of 2C to inhibit its multimerization, so as to inhibit the helicase function, finally achieving the purpose of inhibiting virus replication.

The polypeptide provided by the present invention has high-efficiency and broad-spectrum antiviral activity, which provides a new strategy for the prevention and control of enteroviruses such as EV71, CVA16, CVA4, CVA6, CVA10, CVB3, CVB5 and Echo 11, and also provides a new theoretical basis for accelerating the development of polypeptide and small molecule drugs against human enteroviruses.

### SUMMARY

In view of this, an object of the present invention is to provide use of a preparation inhibiting multimerization of enterovirus protein 2C as a target in the manufacture of a medicament for preventing and/or treating a viral disease;

Another object of the present invention is to provide a broad-spectrum anti-enterovirus polypeptide inhibitor. The inhibitor has a core sequence shown in SEQ ID NO.21. Specifically, the inhibitor has a sequence shown in SEQ ID NO.1 or SEQ ID NO.24, and the sequence containing a cell-penetrating peptide are respectively shown in SEQ ID NO.2 and SEQ ID NO.20.

Another object of the present invention is to provide use of the above polypeptide inhibitor in the manufacture of an enterovirus inhibitor.

In order to realize the above objects of the present invention, the present invention provides the following technical solution:

A broad-spectrum anti-enterovirus polypeptide inhibitor is provided, which has a sequence of:

I.
(X1)E(X2)(X3)(X4)R(X5)(X6)(X7)(X8)(X9)(X10)(X11)
EALFQ

wherein:
X1 is selected from the group consisting of arginine (R), asparagine (N) and lysine (K);
X2 is selected from the group consisting of tyrosine (Y) and arginine (R);
X3 is selected from the group consisting of serine(S), asparagine (N) and arginine (R);
X4 is selected from the group consisting of asparagine (N), arginine (R), threonine (T) and histidine (H);
X5 is selected from the group consisting of serine(S), asparagine (N) and histidine (H);
X6 is selected from the group consisting of alanine (A), asparagine (N) and serine(S);
X7 is selected from the group consisting of isoleucine (I), threonine (T) and valine (V);
X8 is selected from the group consisting of glycine (G) and glutamine (Q);
X9 is selected from the group cFIGURESonsisting of asparagine (N), aspartic acid (D) and alanine (A);
X10 is selected from the group consisting of threonine (T), cysteine (C) and lysine (K);
X11 is selected from the group consisting of isoleucine (I) and leucine (L);
and the sequence is shown in SEQ ID NO.21;
II. a sequence with deletion, addition or substitution of at least one amino acid compared to the sequence in I;
III. a sequence that has at least 50% homology with the amino acid sequence in I or II and inhibits enterovirus activity; and
IV. a complementary sequence to the sequence in I or II or III.

The “amino acid” in the present invention includes natural amino acids or unnatural amino acids. Amino acids commonly known to those skilled in the art are all within the protection scope of the present invention.

The above sequence is preferably: REYN (X4) R (X5) (X6) (X7) (X8) (X9) (X10) (X11) EALFQ, as shown in SEQ ID NO.22; further preferably: REYN (X4) R (X5) (X6) (X7) G (X9) T (X11) EALFQ, as shown in SEQ ID NO.23;

In a specific embodiment of the present invention, the sequence is as shown in SEQ ID NO.1 or SEQ ID NO.24, both of which can be added with a cell-penetrating peptide. A sequence with addition of a cell-penetrating peptide to the sequence shown in SEQ ID NO.1 is shown in SEQ ID NO.2. A sequence with addition of a cell-penetrating peptide to the sequence shown in SEQ ID NO.24 is shown in SEQ ID NO.20;

The protection content of the present invention also includes a polypeptide sequence for inhibiting enterovirus containing the sequence shown in SEQ ID NO.1 or SEQ ID NO.24, and an inhibitor with inhibitory activity on enterovirus obtained by replacing different cell-penetrating sequences, performing polypeptide modification, or designing and modifying unnatural amino acids on the basis of polypeptide RQ (SEQ ID NO.2) or B-RQ (SEQ ID NO.20).

In the present invention, the polypeptide with the sequence shown in any one of SEQ ID NOs.21-23 is a core polypeptide of the present invention, and a polypeptide with addition/deletion of amino acids at the N-terminal, or a polypeptide with modification at the C-terminal, or a D configuration of the polypeptide can be used in the manufacture of an enterovirus inhibitor, or in the manufacture of a medicament for treating or preventing enterovirus infections.

Preferably, the modifications to the above core polypeptide include:
addition of 1-5 amino acids to the N-terminal, deletion of 1-13 amino acids from the N-terminal or modification to the C-terminal, or a D configuration of the polypeptide, all of which have the same inhibitory activity as the core polypeptide. Specifically, for addition of amino acids, amino acids such as S, E, L and I can be added to the N-terminal, for example, the addition of LI dipeptide and SELI tetrapeptide; for amino acids deletion, 1-13 (1, 2, 3, 4, 5, 6, 7, 8, 9, 10, 11, 12 or 13) amino acids can be sequentially deleted from the N-terminal; for modification to the C-terminal, amino acids A (BA) and K, PEG4 (tetrapolyethylene glycol), C16 (palmitic acid), Chol (cholesterol) and the like can be modified to the C-terminal. In specific modifications of the present invention, AK dipeptide is usually added at the C-terminal with or without PEG4, C16, Chol and the like (A\K can be added between PEG4, C16 and Chol), for example, AK-C16, AK-PEG4-K-C16 and AK-Chol. In a specific embodiment of the present invention, the sequence shown in SEQ ID NO.1 is taken as an example to perform the above modifications and changes, which can also be performed on the basis of the sequence shown in SEQ ID NO.24.

The inhibitor of the present invention is obtained by adding a cell-penetrating peptide to the polypeptide with inhibitory activity. The cell-penetrating peptide is added according to conventional methods by, for example, linking a cell-penetrating peptide with the polypeptide with inhibitory activity of the present invention via a linking peptide as, for example, a cell-penetrating peptide+a linking peptide+active polypeptide provided by the present invention. The polypeptide with modification to the C-terminal does not need to be added with a cell-penetrating peptide.

The above sequences obtained by conventional methods in the art are all within the protection scope of the present invention. The conventional methods include but are not limited to artificial synthesis, prokaryotic or eukaryotic expression of recombinant proteins comprising the above proteins.

Use of a broad-spectrum anti-enterovirus polypeptide inhibitor, includes use of a polypeptide containing the sequence shown in SEQ ID NO.1, or a polypeptide with addition/deletion of amino acids at the N-terminal or a polypeptide with modification at the C-terminal, or a D configuration of the polypeptide as mentioned above in the manufacture of an enterovirus inhibitor or in the manufacture of a medicament for treating or preventing enterovirus infection.

In the above use, preferably, the enterovirus includes but is not limited to: the Enterovirus genus of the Picornaviridae family, including human enterovirus (EV), coxsackie A virus (CVA), coxsackie B virus (CVB), echovirus, rhinovirus, poliovirus, etc.

In the above use, a disease caused by the enterovirus infection comprises hand-foot-mouth disease, myocarditis, herpetic angina, aseptic meningitis, encephalitis, viral cold, etc.

Compared with the prior art, the present invention has the following advantages:

The polypeptides and derivatives thereof involved in the present invention can inhibit multimerization of enterovirus 2C, thereby inhibiting its helicase function, and they are a novel type of medicament for treating enterovirus against a new target, showing great significance for antiviral drug resistance.

The polypeptide RQ screened out by the present invention has high-efficiency antiviral activity, which provides a new strategy for the prevention and control of enterovirus, and also provides a new theoretical basis for accelerating the development of polypeptide and small molecule drugs against human enteroviruses. Moreover, the clear antiviral mechanism of the RQ series polypeptides can ensure the safety of their uses and the clarity of approach optimization, which is convenient for further development in the future.

### BRIEF DESCRIPTION OF DRAWINGS

FIG. 1A-C shows the results of the determined cytotoxicity of polypeptides SQ, LQ and RQ;

FIG. 2A-E shows the results of the determined efficiency of polypeptide RQ in inhibiting EV71 in RD, Vero, huh7, and 293T cells;

FIG. 3A-B shows the results of the determined efficiencies of polypeptides LQ and SQ in inhibiting EV71 in Vero cells

FIG. 4 shows the results of the determined efficiency of polypeptide RQ in inhibiting CVA16 in RD cells;

FIG. 5 shows the results of the inhibition of 2C helicase activity of EV71 by polypeptide RQ;

FIG. 6A-B shows the results of the inhibition of 2C helicase activity of EV71 and CVA16 by polypeptide RQ;

FIG. 7 shows the results of the inhibition of the multimerization of EV71 2C protein by polypeptide RQ;

FIG. 8 shows the results of the detected cell-penetrating efficiency of polypeptide RQ;

FIG. 9A-C shows the results of the determined toxicity of polypeptide RQ in various cells;

FIG. 10 shows the results of the determined efficiency of polypeptide RQ in inhibiting CVB3 in RD cells;

FIG. 11 shows the results of the determined efficiency of polypeptide RQ in inhibiting Echo 11 in RD cells;

FIG. 12A-B shows the results of the detected antiviral activity of polypeptide RQ against EV71 in mice;

FIG. 13 shows the results of the detected antiviral activity of the variants of polypeptide RQ;

FIG. 14 shows the results of the detected antiviral activity of the modifiers of polypeptide RQ;

FIG. 15 shows the results of the determined efficiency of polypeptide RQ-DRI in inhibiting EV71 in RD cells;

FIG. 16 shows the results of the detected toxicity of polypeptide B-RQ in RD cells;

FIG. 17A-B shows the results of the determined efficiency of polypeptide B-RQ in inhibiting CVB3 and Echo 11 in RD cells.

### DETAILED DESCRIPTION

The present invention discloses a broad-spectrum antiviral drug for enterovirus and an application thereof (a broad-spectrum anti-enterovirus polypeptide inhibitor targeting enterovirus protein 2C and an application thereof). Those skilled in the art can refer to the content of this application and appropriately improve the process parameters for realization. In particular, it should be noted that all similar replacements and modifications are apparent to those skilled in the art, and they are all considered to be included in the present invention. The polypeptide inhibitor, antiviral drug and application of the present invention have been described through preferred embodiments. Those skilled can apparently make modifications or appropriate changes and combinations to the polypeptide inhibitor, antiviral drug and application described herein without departing from the content, spirit and scope of the present invention, to realize and apply the technology of the present invention.

The present invention takes EV71 virus as an example to verify the inhibitory effect of the polypeptide provided by the present invention. Actually, the present invention designs an inhibitor specifically for enterovirus protein 2C as the target, and the inhibitor of the present invention has effect on any virus with enterovirus protein 2C, such as coxsackie A virus (CVA), coxsackie B virus (CVB), echovirus, rhinovirus and poliovirus. In view of space constraints, no more details.

One of the sequences of the inhibitory proteins designed for enterovirus protein 2C in the present invention is REYNNRSAIGNTIEALFQ as shown in SEQ ID NO.1, which is a core sequence. In order to make it work in vivo, a cell-penetrating peptide is linked to the core protein, and the polypeptide with a cell-penetrating peptide linked has a sequence of YGRKKRRQRRRGSGREYNNRSAIGNTIEALFQ as shown in SEQ ID NO.2, named as polypeptide RQ.

The applicant also designed another two inhibitory polypeptides containing a cell-penetrating peptide for enterovirus protein 2C, which have sequences of:

YGRKKRRQRRRGSGLIREYNNRSAIGNTIEALFQ, SEQ ID NO.3, named as polypeptide LQ; and

YGRKKRRQRRRGSGSELIREYNNRSAIGNTIEALFQ, SEQ ID NO.4, named as polypeptide SQ.

EV71 2C protein has the ability of multimerization, which is crucial for correct helicase function by 2C. Based on the structural composition and sequence features of the 2C multimerization domain, the applicant designed a series of polypeptide sequences for REYNNRSAIGNTIEALFQ (SEQ ID No.1), one of the core sequences necessary for protein 2C multimerization. After screening, the applicant found that the polypeptide RQ has a strong virus-inhibiting ability. It has been proved by experiments that RQ can efficiently enter cells, and can inhibit the correct multimerization of protein 2C and thus the helicase function of protein 2C in vitro. The applicant also conducted structural modifications on the basis of RQ to construct a series of variants, and found that these variants also have good anti-virus ability.

The polypeptides involved in the present invention are shown in Table 1:

TABLE 1

| Peptide | Number | Sequence | Tested | Antiviral(s) | IC₅₀/EC₅₀ | CC₅₀ |
|---|---|---|---|---|---|---|
| Core | SEQ ID No. 1 | REYNNRSAIGNTIEALFQ | / | / | / | / |
| sequence | | | | | | |
| SQ | SEQ ID No. 4 | YGRKKRRQRRRGSGSELIREY | In | EV71 | 10.3 | 134.3 |
| | | NNRSAIGNTIEALFQ | vitro | | μM | μM |
| LQ | SEQ ID No. 3 | YGRKKRRQRRRGSGLIREYNN | In | EV71 | 2.26 | >150 |
| | | RSAIGNTIEALFQ | vitro | | μM | μM |
| RQ | SEQ ID No. 2 | YGRKKRRQRRRGSGREYNNR | In | EV71/CVA16/ | 0.41 | >300 |
| | | SAIGNTIEALFQ | vivo | CVA4/CVA6/ | μM/ | μM |
| | | | | CVA10/CVB3/ | 0.37 | |
| | | | | Echo 11 | | |
| EQ | SEQ ID No. 5 | YGRKKRRQRRRGSGEYNNRS | In | EV71 | 1.83 | |
| | | AIGNTIEALFQ | vitro | | μM | |
| YC | SEQ ID No. 6 | YGRKKRRQRRRGSGYNNRSAI | In | EV71 | 1.96 | / |
| | | GNTIEALFQ | vitro | | μM | |
| NQ | SEQ ID No. 7 | YGRKKRRQRRRGSGNNRSAIG | In | EV71 | 1.90 | / |
| | | NTIEALFQ | vitro | | μM | |
| RSQ | SEQ ID No. 8 | YGRKKRRQRRRGSGRSAIGNT | In | EV71 | 2.60 | / |
| | | IEALFQ | vitro | | μM | |
| SAQ | SEQ ID No. 9 | YGRKKRRQRRRGSGSAIGNTI | In | EV71 | 2.90 | / |
| | | EALFQ | vitro | | μM | |
| AQ | SEQ ID No. 10 | YGRKKRRQRRRGSGAIGNTIE | In | EV71 | 2.99 | / |
| | | ALFQ | vitro | | μM | |
| IQ | SEQ ID No. 11 | YGRKKRRQRRRGSGIGNTIEA | In | EV71 | 1.64 | / |
| | | LFQ | vitro | | μM | |
| GQ | SEQ ID No. 12 | YGRKKRRQRRRGSGGNTIEAL | In | EV71 | 1.78 | / |
| | | FQ | vitro | | μM | |
| NTQ | SEQ ID No. 13 | YGRKKRRQRRRGSGNTIEALF | In | EV71 | 2.28 | / |
| | | Q | vitro | | μM | |
| TQ | SEQ ID No. 14 | YGRKKRRQRRRGSGTIEALFQ | In | EV71 | 1.76 | / |
| | | | vitro | | μM | |
| IEQ | SEQ ID No. 15 | YGRKKRRQRRRGSGIEALFQ | In | EV71 | 2.48 | / |
| | | | vitro | | μM | |
| RQ-PA | SEQ ID No. 16 | REYNNRSAIGNTIEALFQ-βAK- | In | EV71 | 3.58 | / |
| | | C16 | vitro | | μM | |
| RQ-PE | SEQ ID No. 17 | REYNNRSAIGNTIEALFQ-βAK- | In | EV71 | 3.47 | / |
| G4-PA | | PEG4-K-C16 | vitro | | μM | |
| RQ- | SEQ ID No. 18 | REYNNRSAIGNTIEALFQ-βAK- | In | EV71 | 4.25 | / |
| CHOL | | Chol | vitro | | μM | |
| RQ- | SEQ ID No. 19 | AC-qflaeitngiasrnnyergsgrrrq | In | EV71 | 2.05 | / |
| DRI | | rrkkrgy-NH₂ (D-configuration | vitro | | μM | |
| | | amino acid) | | | | |
| B-RQ | SEQ ID No. 20 | YGRKKRRQRRRGSGREYNHR | In | CVB3/ | 0.38 | >75 |
| | | HSVGATLEALFQ | vitro | Echo 11 | μM | μM |
| Core | SEQ ID No. 21 | (X1)E(X2)(X3)(X4)R(X5)(X6) | | | | |
| sequence | | (X7)(X8)(X9)(X10)(X11)EALFQ | | | | |
| | SEQ ID No. 22 | REYN(X4)R(X5)(X6)(X7)(X8) | | | | |
| | | (X9)(X10)(X11)EALFQ | | | | |
| | SEQ ID No. 23 | REYN(X4)R(X5)(X6)(X7)G(X9) | | | | |
| | | T(X11)EALFQ | | | | |
| | SEQ ID No. 24 | REYNHRHSVGATLEALFQ | | | | |

In the polypeptide sequence of the present invention, YGRKKRRQRRR (TAT) is the cell-penetrating peptide, GSG is the linking peptide, and the amino acid sequence of each polypeptide after removal of the cell-penetrating peptide and linking peptide is the sequence or partial sequence of the core polypeptide. The polypeptides having sequences shown in SEQ ID Nos.3 and 4 are the polypeptides of a cell-penetrating peptide+a linking peptide+the core polypeptide having the sequence shown in SEQ ID No.1 with addition of LI and SELI to the N-terminal. The polypeptides having sequences shown in SEQ ID Nos.5-15 are the polypeptides of a cell-penetrating peptide+a linking peptide+the core polypeptide having the sequence shown in SEQ ID No.1 with sequential deletion of 1-12 amino acids from the N-terminal. The polypeptides having sequences shown in SEQ ID Nos.16-18 are the polypeptides of the core polypeptide having the sequence shown in SEQ ID No.1 with modification of amino acids A (BA) and K, PEG4 (tetrapolyethylene glycol), C16 (palmitic acid) and Chol (cholesterol) to the C-terminal. The polypeptide having a sequence shown in SEQ ID No.19 is the D configuration of the core polypeptide of the sequence shown in SEQ ID No.1. The polypeptide having a sequence shown in SEQ ID No.20 is the polypeptides of a cell-penetrating peptide+a linking peptide+the core polypeptide having the sequence shown in SEQ ID No.24;

Negative controls were set for the polypeptides in each example of the present invention, which prove that the core sequences of the polypeptides provided by the present invention have corresponding antiviral efficacy.

The raw materials and reagents used in the polypeptide and application thereof provided by the present invention are all commercially available.

The present invention is further described in conjunction with examples below.

### Example 1: Toxicity of Polypeptides RQ, LQ and SQ in Vero Cells

### 1. Experimental Materials

Vero E6 cells; DMEM medium (Thermo) and serum (Gibco) were purchased from Invitrogen Co., Ltd; CCK-8 reagent (MCE) was purchased from Promoter Company.

Polypeptide SQ was synthesized by Nanjing GenScript Co., Ltd, with a sequence shown in SEQ ID NO.4. Polypeptide LQ was synthesized by Nanjing GenScript Co., Ltd, with a sequence shown in SEQ ID NO.3. Peptide RQ was synthesized by Nanjing GenScript Co., Ltd, with a sequence shown in SEQ ID NO.2.

### 2. Experimental Process

In the anti-virus process, the polypeptide needs to not only inhibit the virus, but also ensure no toxicity to cells. Therefore, this standard was testified by cytotoxicity assay, and the cells without any treatment were used as a control group.

The steps are as follows:

(1) Vero cells were plated in a 96-well plate at 100 μL per well.

(2) When the cells grew to a confluence of 70%-80%, the DMEM medium containing 10% serum was replaced with a DMEM medium containing 2% serum, and a certain concentration gradient of polypeptide RQ or LQ or SQ was added, so that the final concentrations of the polypeptide in the wells were 0.073242 μM, 0.146484 μM, 0.292969 μM, 0.585938 μM, 1.171875 μM, 2.34375 μM, 4.6875 μM, 9.375 μM, 18.75 μM, 37.5 μM, 75 μM and 150 μM, respectively.

(3) The samples were collected 24 h after the addition of the polypeptide, and then 10 μL of live cell detection reagent CCK-8 was added to each well and mixed well.

(4) The plate was placed at 37° C. for 2 h.

(5) The absorbance value at OD450 was detected by a microplate reader.

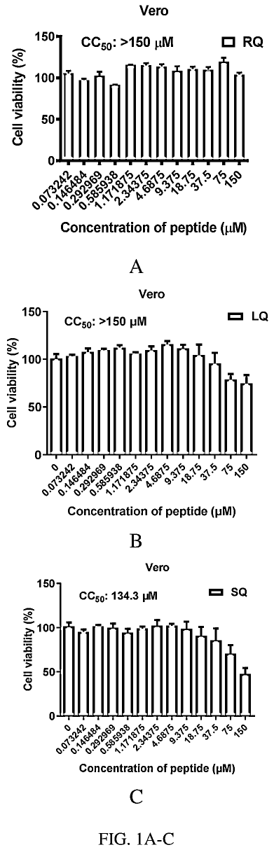

The results are shown in FIG. 1 and Tables 2-4. The CC50 of each polypeptide was calculated taking the cell viability of untreated cells as 100%. RQ had a CC50 of >150 μM (FIG. 1A), LQ had a CC50 of >150 μM (FIG. 1B), and SQ had a CC50 of 134.3 μM (FIG. 1C);

TABLE 2

| Concentration of RQ | | | |
|---|---|---|---|
| polypeptide (μM) | Cell viability (%) | | |
| 0.073242 | 100.6749 | 110.2362 | 105.9618 |
| 0.146484 | 93.81327 | 99.10011 | 98.98763 |
| 0.292969 | 92.68841 | 108.2115 | 106.7492 |
| 0.585938 | 92.23847 | 91.56355 | 90.77615 |
| 1.171875 | 116.0855 | 115.2981 | 115.5231 |
| 2.34375 | 115.8605 | 111.0236 | 119.1226 |
| 4.6875 | 112.7109 | 109.7863 | 118.7852 |
| 9.375 | 117.5478 | 109.3363 | 99.10011 |
| 18.75 | 111.1361 | 115.4106 | 105.7368 |
| 37.5 | 105.6243 | 116.0855 | 108.5489 |
| 75 | 110.0112 | 127.5591 | 120.5849 |
| 150 | 108.2115 | 103.5996 | 100.1125 |

TABLE 3

| Concentration of LQ | | | |
|---|---|---|---|
| polypeptide (μM) | Cell viability (%) | | |
| 0.073242 | 100 | 105.88408 | 97.01806 |
| 0.146484 | 102.8559 | 104.7744 | 101.9913 |
| 0.292969 | 111.8018 | 106.5265 | 104.7619 |
| 0.585938 | 110.1638 | 110.5563 | 108.0087 |
| 1.171875 | 111.3398 | 115.1555 | 110.4762 |
| 2.34375 | 104.8299 | 106.3075 | 107.1861 |
| 4.6875 | 110.8858 | 104.8489 | 112.5108 |
| 9.375 | 114.0277 | 119.5795 | 113.5931 |
| 18.75 | 108.6518 | 115.6373 | 110.3247 |
| 37.5 | 117.0937 | 99.1853 | 97.1429 |
| 75 | 108.2318 | 87.6084 | 90.8658 |
| 150 | 72.1798 | 83.0968 | 80.9091 |

TABLE 4

| Concentration of SQ | | | |
|---|---|---|---|
| polypeptide (μM) | Cell viability (%) | | |
| 0.073242 | 104.2439 | 103.4468 | 96.06877 |
| 0.146484 | 98.04083 | 94.606 | 92.7401 |
| 0.292969 | 101.0204 | 103.1624 | 100.4524 |
| 0.585938 | 94.40455 | 103.9959 | 100.6225 |
| 1.171875 | 90.14603 | 94.06821 | 98.81252 |
| 2.34375 | 96.60283 | 101.1325 | 99.03158 |
| 4.6875 | 105.1932 | 106.7734 | 95.3706 |
| 9.375 | 99.4719 | 102.9859 | 103.7701 |
| 18.75 | 105.7187 | 100.481 | 90.24951 |
| 37.5 | 100.7644 | 90.65433 | 80.59708 |
| 75 | 90.35876 | 95.75345 | 70.30992 |
| 150 | 70.01401 | 80.35016 | 60.9948 |

### Example 2: Determination of Efficiency of Polypeptide RQ in Inhibiting EV71 in RD, Vero, Huh7, And 293T Cells

### 1. Experimental Materials

RD cells, Vero E6 cells, huh7 cells, and 293T cells; DMEM medium (Thermo) and serum (Gibco) were purchased from Invitrogen Co., Ltd; Total RNA extraction kit (Omega) and One step qRT-PCR kit (Takara) were purchased from U-MeBiotech Co., Ltd. The water used in the processes of RNA extraction and qRT-PCR was DEPC water, and the entire experiment was performed in an RNase-free environment.

Polypeptide RQ was synthesized by Nanjing GenScript Co., Ltd, with a sequence shown in SEQ ID NO.2. The cell-penetrating peptide YGRKKRRQRRR (TAT) was used as a control and synthesized by Nanjing GenScript.

### 2. Experimental Process

(1) Different cells were plated in a 24-well plate.

(2) When the cells grew to a confluence of 70%-80%, the DMEM medium containing 10% serum was replaced with a DMEM medium containing 2% serum (the DMEM medium containing 2% serum was added in each well at an amount of 0.5 mL), and 5 μL of EV71 virus of 1×10⁶ PFU/mL was added to each well.

(3) After 1 h, different polypeptides (RQ or control TAT) with final concentrations of 0.3125 μM, 0.625 μM, 1.25 μM, 2.5 μM, and 5 μM were added respectively. The group without polypeptide added was used as a control.

(4) The samples were collected 24 h after the infection of EV71 virus, and RNA was extracted with the total RNA extraction kit.

(5) The supernatant was discarded, then 350 μL of TRK lysate was added to each well, and the plate was put on a shaker for 5 min.

(6) 350 μL of 70% ethanol (DEPC) was added to each well, and then the plate was put on a shaker for 5 min.

(7) The solution in each well was transferred to a RNA extraction column for 1 min of centrifugation at 12000 g.

(8) The solution obtained in the recovery tube was reloaded on the column again for 1 min of centrifugation at 12000 g.

(9) RNA washing buffer 1 was added in the column for 30 s of centrifugation at 12000 g.

(10) RNA washing buffer 2 was added in the column for 1 min of centrifugation at 12000 g.

(11) Step (10) was repeated.

(12) The column alone was centrifuged at 12000 g for 2 min to completely remove the residual RNA washing buffer.

(13) 50 μL of DEPC water was added to the column for 2 min of centrifugation at 12000 g.

(14) 2 μL of RNA sample was subjected to a fluorescence quantitative experiment using the one step qRT-PCR kit.

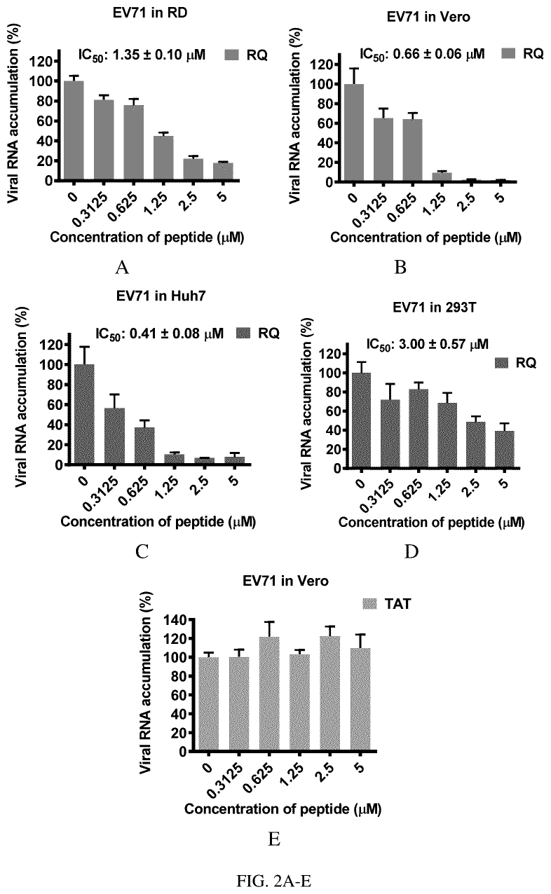

The results are shown in FIG. 2 and Tables 5-9. The results of the determined anti-EV71 effect of polypeptide RQ in different cells show an IC50 of 1.35 μM in RD cells (FIG. 2A), an IC50 of 0.66 μM in Vero cells (FIG. 2B), an IC50 of 0.41 μM in huh7 cells (FIG. 2C), and an IC50 of 3 μM in 293T cells (FIG. 2D). The cell-penetrating peptide control TAT had no anti-EV71 effect in Vero cells (FIG. 2E);

TABLE 5

| Concentration of | | | | | | | | | |
|---|---|---|---|---|---|---|---|---|---|
| RQ (μM) | Percentage of viral RNA in RD cells (%) | | | | | | | | |
| 0 | 98.74564 | 96.68729 | 104.5671 | 82.17592 | 130.0352 | 87.78892 | 84.09811 | 100.7668 | 115.1351 |
| 0.3125 | 75.34828 | 80.42976 | 84.75739 | 79.07512 | 85.1167 | 65.50396 | 105.6539 | 93.32565 | 61.22043 |
| 0.625 | 73.54402 | 85.43979 | 113.5023 | 86.56448 | 56.64673 | 58.60146 | 62.52759 | 60.61082 | 84.55632 |
| 1.25 | 30.05595 | 42.79864 | 38.42442 | 49.01438 | 63.21512 | 36.02987 | 48.88676 | 46.65231 | 50.16218 |
| 2.5 | 14.68721 | 22.11395 | 20.34741 | 13.63269 | 8.986402 | 25.41335 | 28.37401 | 31.44852 | 32.99647 |
| 5 | ND | 15.74388 | 16.39792 | 19.14505 | 16.02361 | 15.46365 | 21.74359 | 23.04845 | 14.24234 |

ND, not detected

TABLE 6

| Concentration of | | | | | | |
|---|---|---|---|---|---|---|
| RQ (μM) | Percentage of viral RNA in Vero cells (%) | | | | | |
| 0 | 82.32617 | 153.297 | 64.37678 | ND | 117.1999 | 82.80011 |
| 0.3125 | 87.78578 | 47.76442 | 71.67834 | 86.67063 | 70.51145 | 25.67804 |
| 0.625 | 46.90411 | 82.11225 | 73.54103 | 51.71642 | 65.58883 | ND |
| 1.25 | 6.624615 | 16.23832 | 12.20634 | 9.196032 | 5.526034 | 7.367869 |
| 2.5 | 2.980446 | 0.826799 | 1.137232 | 3.105048 | 2.98824 | 2.97166 |
| 5 | 2.389376 | 0.957253 | 2.002879 | 1.797167 | 1.955004 | 2.113301 |

ND, not detected

TABLE 7

| Concentration of RQ (μM) | Percentage of viral RNA in Huh7 cells (%) | | |
|---|---|---|---|
| 0 | 135.2764 | 80.15702 | 84.56655 |
| 0.3125 | 52.90425 | 81.62619 | 35.41682 |
| 0.625 | 26.90658 | 51.32333 | 32.9747 |
| 1.25 | 11.6287 | 6.87045 | 12.90771 |
| 2.5 | 7.068214 | 6.953349 | 6.49349 |
| 5 | 0.715564 | 7.820489 | 14.67491 |

TABLE 8

| Concentration of | | | | | | |
|---|---|---|---|---|---|---|
| RQ (μM) | Percentage of viral RNA in 293T cells (%) | | | | | |
| 0 | 140.6661 | 89.65254 | 69.68134 | 86.48279 | 127.9453 | 85.57189 |
| 0.3125 | 66.50167 | 26.53837 | 41.68405 | 142.3522 | 86.73876 | 67.75856 |
| 0.625 | 90.63467 | 82.72855 | 62.9566 | 112.2779 | 72.71821 | 76.02054 |
| 1.25 | 46.14077 | 61.77934 | 42.09579 | 110.8795 | 85.43184 | 64.39499 |
| 2.5 | 50.03924 | 21.79971 | 51.62416 | 51.28562 | 65.19374 | 51.55135 |
| 5 | 23.20045 | ND | 24.63166 | 65.17771 | 46.72863 | 36.78709 |

ND, not detected

TABLE 9

| | |
|---|---|
| Concentration of | |
| TAT (μM) | Percentage of viral RNA in Vero cells (%) |

| | | | |
|---|---|---|---|
| 0 | 91.39502 | 100.2266 | 108.3784 |
| 0.3125 | 115.6133 | 93.74802 | 91.92313 |
| 0.625 | 121.4818 | 94.86778 | 149.0928 |
| 1.25 | 111.6527 | 97.24053 | 100.8214 |
| 2.5 | 125.8799 | 103.6078 | 137.9907 |
| 5 | 136.8661 | 104.0616 | 88.68295 |

### Example 3: Determination of Efficiency of Polypeptides LQ and SQ in Inhibiting EV71 in Vero Cells

### 1. Experimental Materials

Vero E6 cells; DMEM medium (Thermo) and serum (Gibco) were purchased from Invitrogen Co., Ltd; Total RNA extraction kit (Omega) and One step qRT-PCR kit (Takara) were purchased from U-MeBiotech Co., Ltd. The water used in the processes of RNA extraction and qRT-PCR was DEPC water, and the entire experiment was performed in an RNase-free environment.

Polypeptide LQ was synthesized by Nanjing GenScript Co., Ltd, with a sequence shown in SEQ ID NO.3. Polypeptide SQ was synthesized by Nanjing GenScript Co., Ltd, with a sequence shown in SEQ ID NO.4

### 2. Experimental Process

(1) Vero E6 cells were plated in a 24-well plate.

(2) When the cells grew to a confluence of 70%-80%, the DMEM medium containing 10% serum was replaced with a DMEM medium containing 2% serum (the DMEM medium containing 2% serum was added in each well at an amount of 0.5 mL), and 5 μL of EV71 virus of 1×10⁶ PFU/mL was added to each well.

(3) After 1 h, different polypeptides (LQ or SQ) with final concentrations of 0.3125 μM, 0.625 μM, 1.25 μM, 2.5 μM, and 5 μM were added respectively. The group without polypeptide added was used as a control.

(4) The samples were collected 24 h after the infection of EV71 virus, and RNA was extracted with the total RNA extraction kit.

(5) The supernatant was discarded, then 350 μL of TRK lysate was added to each well, and the plate was put on a shaker for 5 min.

(6) 350 μL of 70% ethanol (DEPC) was added to each well, and then the plate was put on a shaker for 5 min.

(7) The solution in each well was transferred to a RNA extraction column for 1 min of centrifugation at 12000 g.

(8) The solution obtained in the recovery tube was reloaded on the column again for 1 min of centrifugation at 12000 g.

(9) RNA washing buffer 1 was added in the column for 30 s of centrifugation at 12000 g.

(10) RNA washing buffer 2 was added in the column for 1 min of centrifugation at 12000 g.

(11) Step (10) was repeated.

(12) The column alone was centrifuged at 12000 g for 2 min to completely remove the residual RNA washing buffer.

(13) 50 μL of DEPC water was added to the column for 2 min of centrifugation at 12000 g.

(14) 2 μL of RNA sample was subjected to a fluorescence quantitative experiment using the one step qRT-PCR kit.

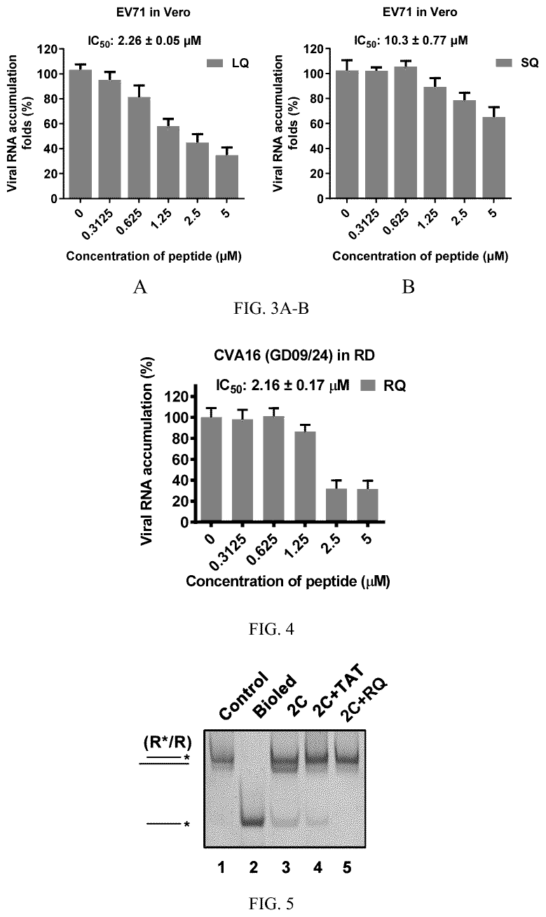

The results are shown in FIG. 3 and Tables 10-11, which indicate that LQ polypeptide had an IC50 of 2.26 μM in Vero cells (FIG. 3A), and SQ polypeptide had an IC50 of 10.3 μM in Vero cells (FIG. 3B).

The above results show that the inhibitory proteins designed for protein 2C had significantly different inhibition efficiency against the virus, where the polypeptide RQ had an IC50 of 0.66 μM in Vero cells, showing significantly higher inhibition efficiency than those of polypeptide LQ and polypeptide SQ;

TABLE 10

| Concentration of LQ (μM) | Percentage of viral RNA in Vero cells (%) | | |
|---|---|---|---|
| 0 | 101.0805 | 100.8891 | 108.1782 |
| 0.3125 | 92.0208 | 91.1729 | 102.4602 |
| 0.625 | 90.7271 | 81.3501 | 71.8857 |
| 1.25 | 60.5336 | 51.4135 | 62.3497 |
| 2.5 | 52.6153 | 41.584 | 40.6992 |
| 5 | 41.9179 | 31.799 | 30.9085 |

TABLE 11

| Concentration of SQ (μM) | Percentage of viral RNA in Vero cells (%) | | |
|---|---|---|---|
| 0 | 94.938 | 111.0771 | 101.7717 |
| 0.3125 | 101.0203 | 100.3849 | 105.3364 |
| 0.625 | 110.1337 | 105.2279 | 100.9028 |
| 1.25 | 90.2836 | 95.7988 | 81.9498 |
| 2.5 | 81.2941 | 82.75 | 71.6192 |
| 5 | 60.1186 | 61.1934 | 74.2715 |

### Example 4: Determination of Efficiency of Polypeptide RQ in Inhibiting CVA16 in RD Cells

### 1. Experimental Materials

RD cells; DMEM medium (Thermo) and serum (Gibco) were purchased from Invitrogen Co., Ltd; Total RNA extraction kit (Omega) and One step qRT-PCR kit (Takara) were purchased from U-MeBiotech Co., Ltd. The water used in the processes of RNA extraction and qRT-PCR was DEPC water, and the entire experiment was performed in an RNase-free environment.

Polypeptide RQ was synthesized by Nanjing GenScript Co., Ltd with a sequence shown in SEQ ID NO.2.

### 2. Experimental Process

(1) RD cells were plated in a 24-well plate.

(2) When the cells grew to a confluence of 70%-80%, the DMEM medium containing 10% serum was replaced with a DMEM medium containing 2% serum (the DMEM medium containing 2% serum was added in each well at an amount of 0.5 mL), and 5 μL of CVA16 virus of 1×10⁶ PFU/mL was added to each well.

(3) After 1 h, different polypeptides with final concentrations of 0.3125 μM, 0.625 μM, 1.25 μM, 2.5 μM, and 5 μM were added respectively. The group without polypeptide added was used as a control.

(4) The samples were collected 24 h after the infection of CVA16 virus, and RNA was extracted with the total RNA extraction kit.

(5) The supernatant was discarded, then 350 μL of TRK lysate was added to each well, and the plate was put on a shaker for 5 min.

(6) 350 μL of 70% ethanol (DEPC) was added to each well, and then the plate was put on a shaker for 5 min.

(7) The solution in each well was transferred to a RNA extraction column for 1 min of centrifugation at 12000 g.

(8) The solution obtained in the recovery tube was reloaded on the column again for 1 min of centrifugation at 12000 g.

(9) RNA washing buffer 1 was added in the column for 30 s of centrifugation at 12000 g.

(10) RNA washing buffer 2 was added in the column for 1 min of centrifugation at 12000 g.

(11) Step (10) was repeated.

(12) The column alone was centrifuged at 12000 g for 2 min to completely remove the residual RNA washing buffer.

(13) 50 μL of DEPC water was added to the column for 2 min of centrifugation at 12000 g.

(14) 2 μL of RNA sample was subjected to a fluorescence quantitative experiment using the one step qRT-PCR kit.

The results of the determined anti-CVA16 effect of polypeptide RQ in RD cells are shown in Table 12 and FIG. 4, which indicate an IC50 of 2.16 μM.

TABLE 12

| Concentration of | | | | | | | | | |
|---|---|---|---|---|---|---|---|---|---|
| RQ (μM) | Percentage of viral RNA in RD cells (%) | | | | | | | | |
| 0 | 78.38927 | 97.34762 | 124.2631 | 53.65574 | 123.7802 | 122.564 | 93.49458 | 77.16421 | 129.3412 |
| 0.3125 | 100.0216 | 148.3965 | 77.29282 | 125.3469 | 101.8237 | 69.03192 | 64.02673 | 86.24284 | 110.1741 |
| 0.625 | 115.4959 | 79.1848 | 91.90599 | 99.67949 | 99.63403 | 80.72403 | 145.8942 | 96.4369 | ND |
| 1.25 | 83.98713 | 74.20358 | 80.83334 | 111.5916 | 81.89601 | 63.92798 | 62.81053 | 100.5664 | 117.5637 |
| 2.5 | 68.28515 | 40.30227 | 61.91291 | 27.90528 | 46.74052 | 21.57059 | 4.815019 | 2.49757 | 13.56626 |
| 5 | 38.35756 | 89.90899 | 32.58479 | 25.69166 | 15.84122 | 34.43394 | 18.04668 | 7.616083 | 21.36344 |

ND, not detected

### Example 5: Inhibition of EV71 2C Helicase Activity by RQ

### 1. Experimental Materials

Baculovirus for fusion expression of MBP-EV71 2C protein; Spodoptera frugiperda cells (Sf9) were obtained from China Center for Type Culture Collection (CCTCC), culture medium (SF-HM) was purchased from Beijin Compamy, maltose binding protein (MBP) filler was purchased from NEB, Amicon Mltra-30KDa (ultrafiltration tube) was purchased from Millipore; binding buffer (pH 7.4): 20 mM Tris-HCl (pH 7.4), 0.5 M EDTA, 200 mM NaCl, 10 mM B-mercaptoethanol, anhydrous ethanol of 5% by volume, and glycerol of 10% by volume. Elution buffer: 10 mM maltose solution. 50 mM HEPES solution at pH 7.5.

HEX fluorescently labeled RNA single strand at a length of 42 nt, and a RNA single strand at a length of 54 nt complementary to the HEX labeled RNA strand.

Polypeptide RQ was synthesized by Nanjing GenScript Co., Ltd with a sequence shown in SEQ ID NO.2.

### 2. Experimental Process

2.1 In vitro expression and purification of EV71 2C protein

(1) 1 mL of baculovirus expressing MBP-EV71 2C protein was added to each of 6 flasks (T75) of Sf9 cells with a density of 80-90%. The flasks were placed at 27.5° C. for 3 days of infection. When the cells presented with obvious symptoms of virus infection (the cells became larger and rounder, and a large number of them were suspended), Sf9 cells were blown off with the original medium, and then centrifuged at 1000 g for 5 min. The supernatant was discarded, and the cells were resuspended with 15 mL of the binding buffer for purifying MBP fusion protein.

(2) Sf9 cells were broken by ultrasonication (250 W, 15-20 min) to be transparent, then aliquoted into 1.5 mL centrifuge tubes, and centrifuged at 12000 g and 4° C. for 15 min. Then the supernatant was transferred into a 15 mL centrifuge tube and placed on ice.

(3) A chromatography column was added with 2-3 mL of Amylose Resin, washed with 30 mL of ddH2O, and then added with 30 mL of the binding buffer to equilibrate the filler. It should be noted that air bubbles were not allowed to exist in the filler during the washing process.

(4) The supernatant containing the target protein was slowly added into the equilibrated column, and a constant flow pump was set at a flow rate of 50 or 60, so that the flow rate of the protein sample was 7-8 s/drop. 15 mL of supernatant was loaded 3 times.

(5) After binding, the filler was washed with 100 mL of the binding buffer at a flow rate of the constant flow pump of 130 to wash off impurities.

(6) After washing, the filler was eluted with 10 mM maltose eluent at a flow rate of the constant flow pump of 10. The collected eluate (containing the target protein) was added into a 30 KD ultrafiltration tube and centrifuged at 7200 g and 4° C. for ultrafiltration to concentrate the target protein (about 200-300 μL, with a concentration of about 1 mg/mL).

(7) After the ultrafiltration was completed, the buffer system of the target protein was replaced with 50 mM HEPES at pH 7.5 (by ultrafiltration with HEPES-KOH for 3-4 times).

(8) 2 μL of the purified protein sample was subjected to SDS-PAGE electrophoresis, and the remaining protein was stored at −80° C. for later use.

EV71 2C protein labeled with MBP was successfully purified.

2.2 Inhibition of helicase activity of EV71 2C protein by RQ in vitro

(1) A strand labeled with HEX at a concentration of 0.2 pmol/μL was added with a complementary strand RNA at the same concentration to prepare a HEX-labeled double-stranded dsRNA substrate by annealing.

(2) Annealing process: the reaction system was kept at 75° C. for 3 min, cooled to 25° C. at a rate of 1° C. per minute, and then kept at 25° C. for 2 min.

(3) The target protein and the double-stranded substrate were prepared according to the standard unwinding experiment reaction system. 5 μg of polypeptide RQ and control TAT were added respectively, and single and double strands controls were set. The single strand sample was boiled at 75° C. for 3 min and then placed on ice for 2 min.

(4) The prepared system was mixed well and then placed at 37° C. for 50 min of reaction.

(5) A mixture obtained after the reaction was subjected to electrophoresis.

(6) Finally, Typhoon 9500 was used for direct scan to obtain a HEX signal.

During electrophoresis, the single strand ran faster than the double strand. Therefore, if the MBP-2C protein had helicase activity, it can unwind the double-stranded dsRNA substrate to release a single-stranded RNA, and then the lane would show two bands, upper and lower. The single-stranded RNA (lane 2) prepared by boiling at 75° C. was used as a positive control. The reaction without protein added (lane 1) was used as a negative control. As shown in lane 3 of FIG. 5, EV71 2C had helicase activity and can unwind the double-stranded dsRNA substrate; whereas the addition of RQ inhibited the helicase activity of 2C (lane 5), and the control TAT did not affect the helicase activity of 2C (lane 4). The above results indicate that RQ can indeed inhibit the helicase function of EV71 2C.

### Example 6: Inhibition of 2C helicase activity of EV71 and CVA16 by RQ

### 1. Experimental Materials

Purified MBP-EV71 2C protein; baculovirus for fusion expression of MBP-CVA16 2C protein; Spodoptera frugiperda cells (Sf9) was obtained from China Center for Type Culture Collection (CCTCC), culture medium (SF-HM) was purchased from Beijin Company, maltose-binding protein (MBP) filler was purchased from NEB, Amicon Mltra-30KDa (ultrafiltration tube) was purchased from Millipore; binding buffer (pH 7.4): 20 mM Tris-HCl (pH 7.4), 0.5 M EDTA, 200 mM NaCl, 10 mM B-mercaptoethanol, anhydrous ethanol of 5% by volume, and glycerol of 10% by volume. Elution buffer: 10 mM maltose solution. 50 mM HEPES solution at pH 7.5.

HEX fluorescently labeled RNA single strand at a length of 42 nt, and a RNA single strand at a length of 54 nt complementary to the HEX labeled RNA strand.

Polypeptide RQ was synthesized by Nanjing GenScript Co., Ltd with a sequence shown in SEQ ID NO.2.

### 2. Experimental Process

### 2.1 In Vitro Expression and Purification of CVA16 2C Protein

(1) 1 mL of baculovirus expressing MBP-CVA16 2C protein was added to each of 6 flasks (T75) of Sf9 cells with a density of 80-90%. The flasks were placed at 27.5° C. for 3 days of infection. When the cells presented with obvious symptoms of virus infection (the cells became larger and rounder, and a large number of them were suspended), Sf9 cells were blown off with the original medium, and then centrifuged at 1000 g for 5 min. The supernatant was discarded, and the cells were resuspended with 15 mL of the binding buffer for purifying MBP fusion protein.

(2) Sf9 cells were broken by ultrasonication (250 W, 15-20 min) to be transparent, then aliquoted into 1.5 mL centrifuge tubes, and centrifuged at 12000 g and 4° C. for 15 min. Then the supernatant was transferred into a 15 mL centrifuge tube and placed on ice.

(3) A chromatography column was added with 2-3 mL of Amylose Resin, washed with 30 mL of ddH2O, and then added with 30 mL of the binding buffer to equilibrate the filler. It should be noted that air bubbles were not allowed to exist in the filler during the washing process.

(4) The supernatant containing the target protein was slowly added into the equilibrated column, and a constant flow pump was set at a flow rate of 50 or 60, so that the flow rate of the protein sample was 7-8 s/drop. 15 mL of supernatant was loaded 3 times.

(5) After binding, the filler was washed with 100 mL of the binding buffer at a flow rate of the constant flow pump of 130 to wash off impurities.

(6) After washing, the filler was eluted with 10 mM maltose eluent at a flow rate of the constant flow pump of 10. The collected eluate (containing the target protein) was added into a 30 KD ultrafiltration tube and centrifuged at 7200 g and 4° C. for ultrafiltration to concentrate the target protein (about 200-300 μL, with a concentration of about 1 mg/mL).

(7) After the ultrafiltration was completed, the buffer system of the target protein was replaced with 50 mM HEPES at pH 7.5 (by ultrafiltration with HEPES-KOH for 3-4 times).

(8) 2 μL of the purified protein sample was subjected to SDS-PAGE electrophoresis, and the remaining protein was stored at −80° C. for later use.

EV71 2C protein labeled with MBP was successfully purified.

### 2.2 Inhibition of Helicase Activity of EV71 and CVA16 2C Proteins by RQ In Vitro

(1) A strand labeled with HEX at a concentration of 0.2 pmol/μL was added with a complementary strand RNA at the same concentration to prepare a HEX-labeled double-stranded dsRNA substrate by annealing.

(2) Annealing process: the reaction system was kept at 75° C. for 3 min, cooled to 25° C. at a rate of 1° C. per minute, and then kept at 25° C. for 2 min.

(3) The target protein and the double-stranded substrate were prepared according to the standard unwinding experiment reaction system. 5 μg of polypeptide RQ and control TAT were added respectively, and single and double strands controls were set. The single strand sample was boiled at 75° C. for 3 min and then placed on ice for 2 min.

(4) The prepared system was mixed well and then placed at 37° C. for 50 min of reaction.

(5) A mixture obtained after the reaction was subjected to electrophoresis.

(6) Finally, Typhoon 9500 was used for direct scan to obtain a HEX signal.

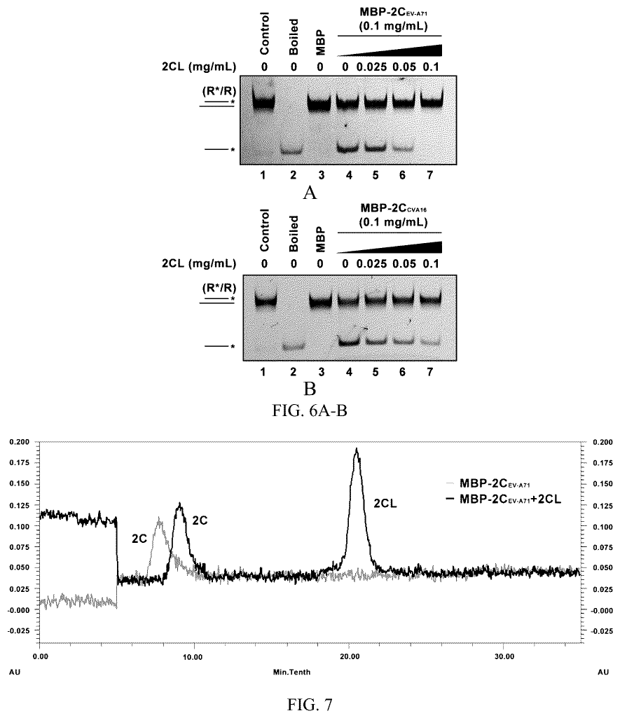

As shown in FIG. 6A, RQ inhibited the helicase activity of EV71 2C in a dose-dependent manner; and as shown in FIG. 6B, RQ inhibited the helicase activity of CVA16 2C in a dose-dependent manner.

### Example 7: Inhibition of Multimerization of EV71 2C Protein by RQ

### 1. Experimental Materials

Purified MBP-EV71 2C protein; Superdex 200 Increase 10/300 GL chromatography column was purchased from GE Healthcare Co., Ltd; Amicon Ultra centrifugal filters were purchased from Merck Co., Ltd; BioLogic DuoFlow system was purchased from Bio-Rad Co., Ltd; 50 mM HEPES-KOH (pH 8.5).

Polypeptide RQ was synthesized by Nanjing GenScript Co., Ltd with a sequence shown in SEQ ID NO.2.

### 2. Experimental Process

(1) The purified MBP-EV71 2C was concentrated to 1 mg/mL with Amicon Ultra centrifugal filters.

(2) The concentrated protein 2C was mixed with 20 μM polypeptide RQ for 1 h of incubation on ice, and the incubation of 2C with ddH2O of the same volume was set as a control.

(3) The above samples were equilibrated with 50 mM HEPES-KOH (pH 8.5), and then loaded onto a Superdex 200 Increase 10/300 GL chromatography column. The flow rate was controlled to be 1 mL/min by the BioLogic DuoFlow system.

(4) The duration of the protein passing through the chromatography column was recorded by ultraviolet (UV) signal, and changes in protein molecular weight were analyzed.

As shown in FIG. 7, in a case that protein 2C was only co-incubated with ddH₂O, 2C formed into a polymer, which was eluted from the system rapidly (light-colored line) with a peak elution time of 8 min (light-colored peak on the left); in a case that RQ was co-incubated with 2C (dark-colored line), the peak elution time of 2C polymer changed obviously (dark-colored peak on the left), and the dark-colored peak on the right represents free polypeptide RQ. The above results indicate that RQ in co-incubation with 2C inhibited the formation of 2C polymer.

### Example 8: Determination of Cell-Penetrating Efficiency of Polypeptide RQ

### 1. Materials

MEM medium (Thermo) and serum (Gibco) were purchased from Invitrogen Co., Ltd, immunofluorescence dish (NEST) was purchased from Promoter Company, PBS, DAPI and paraformaldehyde were purchased from Diyue Chuangxin Company.

Polypeptide RQ was synthesized by Nanjing GenScript Co., Ltd with a sequence shown in SEQ ID NO.2.

### 2. Experimental Process

The experiment was performed with two groups. In order to avoid the impact of adding EV71 virus on the polypeptide entering cells, in one group of the experiment, EV71 virus was added before the addition of polypeptide RQ, while in the other group, no virus was added before the addition of the polypeptide, and each group was set with a negative control.

An immunofluorescence experiment was conducted according to the following steps:

(1) 1 mL of RD cells was added in an immunofluorescence special dish, and cells were collected when grew to a confluence of 30%.

(2) The culture medium was discarded, and the residual culture medium was washed off by washing with 1 mL of PBS of 0.01 mol/L and pH 7.4 three times, 5 min each time.

(3) 4% paraformaldehyde solution was prepared by dissolving 4 g of paraformaldehyde in 100 mL of PBS. 1 mL of the prepared 4% paraformaldehyde was added to each dish for 5 min of reaction to fix the cells.

(4) The 4% paraformaldehyde was discarded, and then the residual paraformaldehyde was washed off by washing with 1 mL of PBS of 0.01 mol/L and pH 7.4 three times, 5 min each time.

(5) 1 mg/mL DAPI solution was diluted to 100 ng/mL with PBS, and then added to the dish for 15 min of reaction.

(6) The reaction solution was discarded, and then the residual reaction solution was washed off by washing with 1 mL of PBS of 0.01 mol/L and pH 7.4 three times, 5 min each time.

(7) The dish was placed under a fluorescence microscope for observation.

The fluorescently labeled (FITC) polypeptide was detected for its cell-penetrating efficiency in RD cells. Two groups of experiments were set up. The first group was an untreated control group, which was added with FITC-RQ. The second group was an EV71-infected group, which was added with FITC-RQ after EV71 infection. The two groups of experiments were performed simultaneously, with a virus MOI of 0.1 and a concentration of the polypeptide added of 1 μM. The samples were collected 12 h after the addition of the polypeptide, and the cells were fixed and subjected to an immunofluorescence experiment. The results show that the polypeptide can enter the cells with or without infection, showing a good cell-penetrating ability.

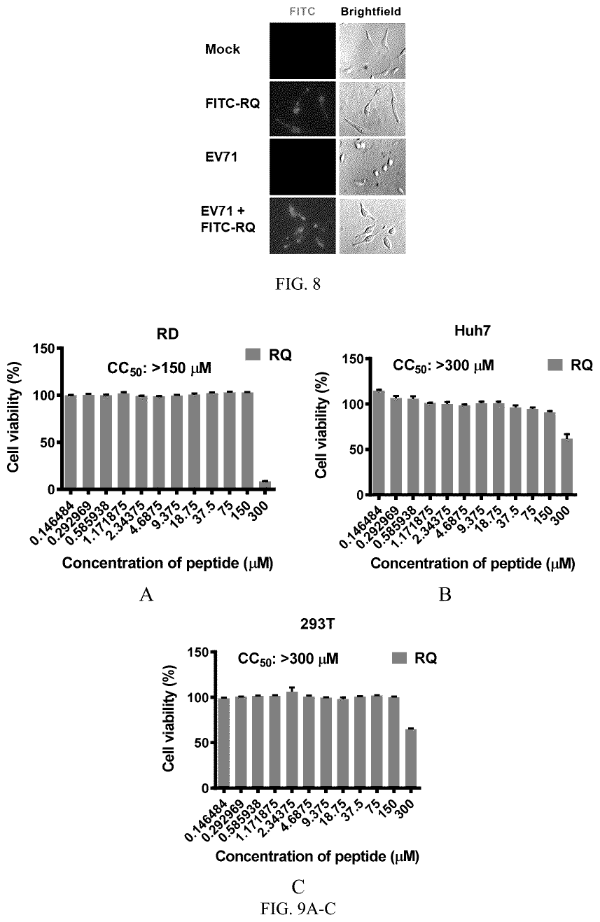

As shown in FIG. 8, the polypeptide can be observed entering the cells with or without virus added, proving that polypeptide RQ had a good cell-penetrating ability.

### Example 9: Toxicity Assay of Polypeptide RQ in Various Cells

### 1. Experimental Materials

RD cells, Huh7 cells, and 293T cells; DMEM medium (Thermo) and serum (Gibco) were purchased from Invitrogen Co., Ltd; CCK-8 reagent (MCE) was purchased from Promoter Company.

Polypeptide RQ was synthesized by Nanjing GenScript Co., Ltd with a sequence shown in SEQ ID NO.2.

### 2. Experimental Process

(1) Different cells were plated in a 96-well plate at 100 μL per well.

(2) When the cells grew to a confluence of 70%-80%, the DMEM medium containing 10% serum was replaced with a DMEM medium containing 2% serum, and a certain concentration gradient of polypeptide RQ was added, so that the final concentrations of the polypeptide in the wells were 0.073242 μM, 0.146484 μM, 0.292969 μM, 0.585938 μM, 1.171875 μM, 2.34375 μM, 4.6875 μM, 9.375 μM, 18.75 μM, 37.5 μM, 75 μM, 150 μM, and 300 μM, respectively.

(3) The samples were collected 24 h after the addition of the polypeptide, and then 10 μL of live cell detection reagent CCK-8 was added to each well and mixed well.

(4) The plate was placed at 37° C. for 2 h.

(5) The absorbance value at OD450 was detected by a microplate reader.

The results are shown in FIG. 9 and Tables 13-15. The CC50 of each RQ in different cells was calculated taking the cell viability of untreated cells as 100%. CC50 was >150 μM in RD cells (FIG. 9A), CC50 was >300 μM in Huh cells (FIG. 9B), and CC50 was >300 μM in 293T cells (FIG. 1C).

TABLE 13

| Concentration of RQ | | | |
|---|---|---|---|
| polypeptide (μM) | Viability of RD cell (%) | | |
| 0.073242 | 99.7786 | 100.3321 | 99.8893 |
| 0.146484 | 99.22509 | 102.3247 | 99.94465 |
| 0.292969 | 100.7749 | 100.2214 | 99.16974 |
| 0.585938 | 98.94834 | 103.5978 | 102.7122 |
| 1.171875 | 99.61255 | 99.00369 | 99.22509 |
| 2.34375 | 98.78229 | 99.22509 | 98.50554 |
| 4.6875 | 98.33948 | 99.44649 | 100.8856 |
| 9.375 | 98.39483 | 100.7196 | 102.6568 |
| 18.75 | 100.6089 | 103.4317 | 102.4354 |
| 37.5 | 101.4945 | 103.9852 | 103.5424 |
| 75 | 102.1033 | 103.5978 | 102.9889 |
| 150 | 8.726937 | 8.782288 | 8.00738 |

TABLE 14

| Concentration of RQ | | | |
|---|---|---|---|
| polypeptide (μM) | Viability of Huh7 cell (%) | | |
| 0.073242 | 113.2507 | 113.9801 | 116.7518 |
| 0.146484 | 110.6978 | 106.6132 | 101.8721 |
| 0.292969 | 110.9895 | 104.4979 | 102.018 |
| 0.585938 | 101.2157 | 100.7051 | 101.5074 |
| 1.171875 | 104.3521 | 97.20399 | 99.02747 |
| 2.34375 | 98.73572 | 100.1945 | 96.03696 |
| 4.6875 | 98.00632 | 103.5497 | 101.2886 |
| 9.375 | 103.4768 | 101.4345 | 97.71456 |
| 18.75 | 100.851 | 93.41114 | 94.28641 |
| 37.5 | 95.0158 | 97.05811 | 92.02529 |
| 75 | 90.93119 | 93.26526 | 88.08655 |
| 150 | 65.84002 | 67.73645 | 51.47095 |

TABLE 15

| Concentration of RQ | | | |
|---|---|---|---|
| polypeptide (μM) | Viability of Huh7 cell (%) | | |
| 0.073242 | 98.39022 | 100.4267 | 97.86656 |
| 0.146484 | 100.6594 | 100.8922 | 100.9503 |
| 0.292969 | 100.7758 | 102.4631 | 100.8922 |
| 0.585938 | 100.4849 | 102.8123 | 101.6486 |
| 1.171875 | 99.67029 | 115.4383 | 103.5687 |
| 2.34375 | 99.08844 | 100.3685 | 102.8704 |
| 4.6875 | 98.56478 | 99.96121 | 100.0776 |
| 9.375 | 99.67029 | 99.96121 | 94.02638 |
| 18.75 | 100.2521 | 99.90303 | 101.8231 |
| 37.5 | 101.8231 | 101.1249 | 102.9286 |
| 75 | 101.5322 | 98.97207 | 99.49573 |
| 150 | 66.56323 | 63.82855 | 64.4104 |

### Example 10: Determination of Efficiency of Polypeptide RQ in Inhibiting CVB3 in RD Cells

### 1. Experimental Materials

RD cells; DMEM medium (Thermo) and serum (Gibco) were purchased from Invitrogen Co., Ltd; Total RNA extraction kit (Omega) and One step qRT-PCR kit (Takara) were purchased from U-MeBiotech Co., Ltd. The water used in the processes of RNA extraction and qRT-PCR was DEPC water, and the entire experiment was performed in an RNase-free environment.

Polypeptide RQ was synthesized by Nanjing GenScript Co., Ltd with a sequence shown in SEQ ID NO.2.

### 2. Experimental Process

(1) RD cells were plated in a 24-well plate.

(2) When the cells grew to a confluence of 70%-80%, the DMEM medium containing 10% serum was replaced with a DMEM medium containing 2% serum (the DMEM medium containing 2% serum was added in each well at an amount of 0.5 mL), and 5 μL of CVB3 virus of 1×10⁶ PFU/mL was added to each well.

(3) After 1 h, the polypeptides with final concentrations of 0.3125 μM, 0.625 μM, 1.25 μM, 2.5 μM, and 5 μM were added respectively. The group without polypeptide added was used as a control.

(4) The samples were collected 24 h after the infection of CVB3 virus, and RNA was extracted with the total RNA extraction kit.

(5) The supernatant was discarded, then 350 μL of TRK lysate was added to each well, and the plate was put on a shaker for 5 min.

(6) 350 μL of 70% ethanol (DEPC) was added to each well, and then the plate was put on a shaker for 5 min.

(7) The solution in each well was transferred to a RNA extraction column for 1 min of centrifugation at 12000 g.

(8) The solution obtained in the recovery tube was reloaded on the column again for 1 min of centrifugation at 12000 g.

(9) RNA washing buffer 1 was added in the column for 30 s of centrifugation at 12000 g.

(10) RNA washing buffer 2 was added in the column for 1 min of centrifugation at 12000 g.

(11) Step (10) was repeated.

(12) The column alone was centrifuged at 12000 g for 2 min to completely remove the residual RNA washing buffer.

(13) 50 μL of DEPC water was added to the column for 2 min of centrifugation at 12000 g.

(14) 2 μL of RNA sample was subjected to a fluorescence quantitative experiment using the one step qRT-PCR kit.

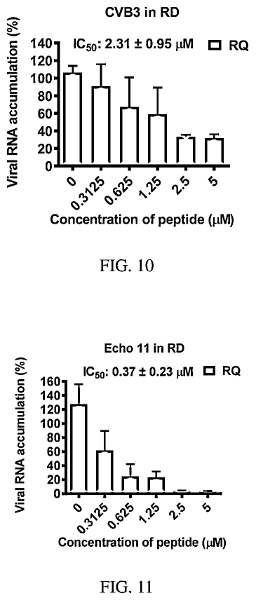

The determination results of the anti-CVB3 effect of polypeptide RQ in RD cells are shown in Table 16 and FIG. 10, which indicate an IC50 of 2.31 μM.

TABLE 16

| Concentration of SQ (μM) | Percentage of viral RNA in Vero cells (%) | | |
|---|---|---|---|
| 0 | 106.8374 | 119.2124 | 93.16261 |
| 0.3125 | 62.25856 | 140.3936 | 70.09917 |
| 0.625 | 30.54633 | 134.359 | 36.93837 |
| 1.25 | 119.4583 | 30.84876 | 26.36559 |
| 2.5 | 35.87058 | 29.1883 | 35.07889 |
| 5 | 31.34734 | 39.17466 | 25.33601 |

### Example 11: Determination of Efficiency of Polypeptide RQ in Inhibiting Echo 11 in RD Cells

### 1. Experimental Materials

RD cells; DMEM medium (Thermo) and serum (Gibco) were purchased from Invitrogen Co., Ltd; Total RNA extraction kit (Omega) and One step qRT-PCR kit (Takara) were purchased from U-MeBiotech Co., Ltd. The water used in the processes of RNA extraction and qRT-PCR was DEPC water, and the entire experiment was performed in an RNase-free environment.

Polypeptide RQ was synthesized by Nanjing GenScript Co., Ltd with a sequence shown in SEQ ID NO.2.

### 2. Experimental Process

(1) RD cells were plated in a 24-well plate.

(2) When the cells grew to a confluence of 70%-80%, the DMEM medium containing 10% serum was replaced with a DMEM medium containing 2% serum (the DMEM medium containing 2% serum was added in each well at an amount of 0.5 mL), and 5 μL of Echo 11 virus of 1×10⁶ PFU/mL was added to each well.

(3) After 1 h, the polypeptides with final concentrations of 0.3125 μM, 0.625 μM, 1.25 μM, 2.5 μM, and 5 μM were added respectively. The group without polypeptide added was used as a control.

(4) The samples were collected 24 h after the infection of Echo 11 virus, and RNA was extracted with the total RNA extraction kit.

(5) The supernatant was discarded, then 350 μL of TRK lysate was added to each well, and the plate was put on a shaker for 5 min.

(6) 350 μL of 70% ethanol (DEPC) was added to each well, and then the plate was put on a shaker for 5 min.

(7) The solution in each well was transferred to a RNA extraction column for 1 min of centrifugation at 12000 g.

(8) The solution obtained in the recovery tube was reloaded on the column again for 1 min of centrifugation at 12000 g.

(9) RNA washing buffer 1 was added in the column for 30 s of centrifugation at 12000 g.

(10) RNA washing buffer 2 was added in the column for 1 min of centrifugation at 12000 g.

(11) Step (10) was repeated.

(12) The column alone was centrifuged at 12000 g for 2 min to completely remove the residual RNA washing buffer.

(13) 50 μL of DEPC water was added to the column for 2 min of centrifugation at 12000 g.

(14) 2 μL of RNA sample was subjected to a fluorescence quantitative experiment using the one step qRT-PCR kit.

The determination results of the anti-Echo 11 effect of polypeptide RQ in RD cells are shown in Table 17 and FIG. 11, which indicate an IC50 of 0.37 μM.

TABLE 17

| Concentration of SQ (μM) | Percentage of viral RNA in Vero cells (%) | | |
|---|---|---|---|
| 0 | 144.9111 | 165.5089 | 72.39416 |
| 0.3125 | 117.2316 | 36.60896 | 30.65337 |
| 0.625 | 59.40357 | 6.90746 | 7.106392 |
| 1.25 | 33.44803 | 29.3462 | 7.071973 |
| 2.5 | 1.051358 | 5.505496 | 3.538126 |
| 5 | 4.488181 | 1.195551 | 2.309541 |

### Example 12: Detection of Antiviral Activity of Polypeptide RQ on EV71 in Mice

### 1. Experimental Materials

Newborn 1-day-old ICR suckling mice. Polypeptide RQ was synthesized by Nanjing GenScript Co., Ltd with a sequence shown in SEQ ID NO.2.

### 2. Experimental Process

(1) Twenty-seven 1-day-old ICR suckling mice were randomly divided into 3 groups; one group of 10 suckling mice was challenged with virus and injected with the same amount of PBS (vehicle) as a positive control, one group of 9 suckling mice was challenged with virus and then injected with RQ, and one group of 8 suckling mice was not challenged with virus and not administered with drugs as a negative control. These 19 suckling mice were challenged with EV71 at a dose of 107 PFU by intraperitoneal injection.

(2) Simultaneously with the challenge, one group was intraperitoneally injected with polypeptide RQ at 20 mg/kg as a treatment group, and the other group was injected with the same amount of PBS as a control group.

(3) The polypeptide and PBS were injected every 12 h until the 7th day after the challenge.

(4) The clinical symptoms and death of suckling mice were observed until the 21st day.

(5) The clinical symptoms were evaluated by a clinical scoring system: 0 point: healthy, 1 point: exhibiting slow and hunchbacked movements, 2 points: weak in one limb, 3 points: one limb paralyzed, 4 points: two limbs paralyzed, and 5 points: died.

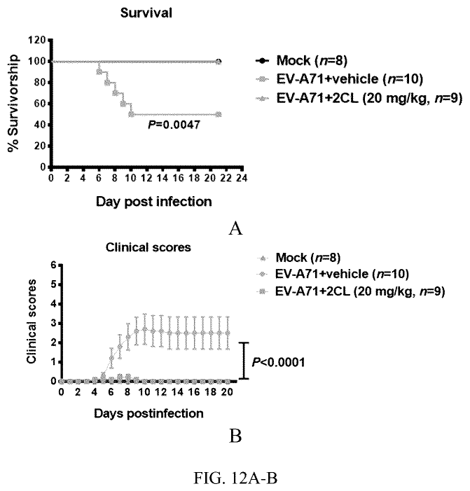

The results are shown in FIG. 12A, which indicate that all the suckling mice in the negative control group (Mock) survived, 5 suckling mice in the group with challenge but no drug administration died on the 10th day, showing a mortality rate of 50%, while all the suckling mice in the group with RQ administration survived. As shown in FIG. 12B, the group with challenge but no drug administration had a significantly higher clinical score than that of the group with drug administration after challenge. The above results indicate that RQ can effectively treat suckling mice infected with a lethal dose of EV71 and prevent them from dying.

### Example 13: Detection of Antiviral Activity of Polypeptide RQ Variants

### 1. Experimental Materials

Polypeptides EQ (shown in SEQ ID NO.5), YQ (shown in SEQ ID NO.6), NQ (shown in SEQ ID NO.7), RSQ (shown in SEQ ID NO.8), SAQ (shown in SEQ ID NO.9), AQ (shown in SEQ ID NO.10), IQ (shown in SEQ ID NO.11), GQ (shown in SEQ ID NO.12), NTQ (shown in SEQ ID NO.13), TQ (shown in SEQ ID NO.14), and IEQ (shown in SEQ ID NO.15). The sequences were all commercially synthesized. CCK-8 reagent (MCE) was purchased from Promoter Company.

### 2. Experimental Process

(1) RD cells were plated in a 96-well plate at 100 μL per well.

(2) When the cells grew to a confluence of 70%-80%, the DMEM medium containing 10% serum was replaced with a DMEM medium containing 2% serum.

(3) The polypeptide drug was gradiently diluted with the DMEM containing 2% FBS into concentrations of 0.15625 μM, 0.3125 μM, 0.625 μM, 1.25 μM, 2.5 μM, and 5 μM, and then added to a new 96-well plate at 100 μL per well, with 3 replicate wells for each concentration.

(4) The diluted virus was added to the above wells at 100 μL per well. The wells with no drug and no virus added and the wells with no drug but virus added were set as controls respectively, with a final concentration of virus of 0.1 MOI.

(4) The mixture was transferred into the 96-well plate plated with cells for another 24 h of culture, and the inhibitory activity of the polypeptide on the virus was determined by a CCK8 kit.

(5) The inhibition rates of different concentrations of the polypeptides on virus infection were calculated according to a calculation formula of: inhibition rate of polypeptide=(well with drug-well with virus)×100%/(well with no drug-well with virus).

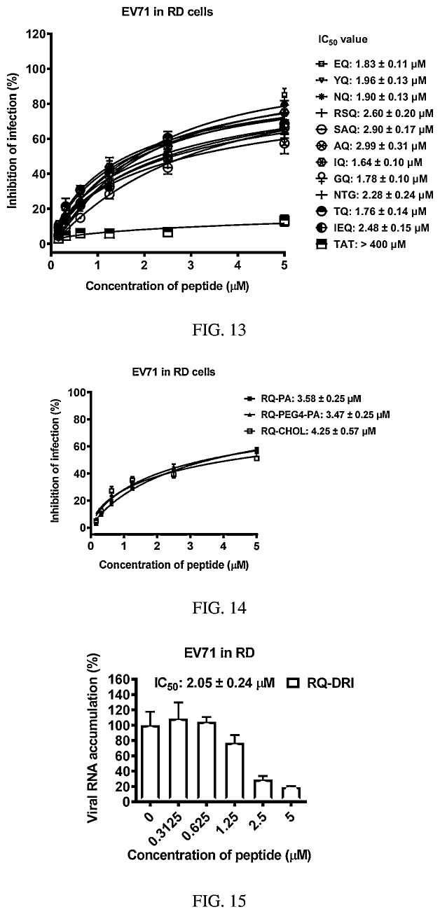

As shown in Tables 18-21 and FIG. 13, the determination of the inhibitory activity of the polypeptides on the virus by a CCK8 method indicates that EQ had an IC50 of 1.83 μM, YQ had an IC50 of 1.96 μM, NQ had an IC50 of 1.90 μM, RSQ had an IC50 of 2.60 μM, SAQ had an IC50 of 2.90 μM, AQ had an IC50 of 2.99 μM, IQ had an IC50 of 1.64 μM, GQ had an IC50 of 1.78 μM, NTG had an IC50 of 2.28 μM, TQ had an IC50 of 1.76 μM, IEQ had an IC50 of 2.48 μM, and control TAT had no antiviral activity.

TABLE 18

| Concentration of | Inhibition rate on EV71 virus (%) | | | | | | | | |
|---|---|---|---|---|---|---|---|---|---|
| polypeptide (μM) | EQ | | | YQ | | | NQ | | |
| 0.15625 | 6.88487 | 4.67462 | 10.9434 | 5.8298 | 7.32961 | 11.0089 | 4.27801 | 5.36773 | 8.33654 |
| 0.3125 | 10.2126 | 12.8194 | 16.4536 | 15.102 | 10.7124 | 13.3881 | 11.5056 | 17.809 | 19.7228 |
| 0.625 | 21.5745 | 20.64 | 25.9399 | 23.6157 | 20.9642 | 29.1606 | 16.7886 | 21.2091 | 24.9365 |
| 1.25 | 31.5814 | 30.7778 | 32.9503 | 30.9365 | 28.1124 | 32.0401 | 41.6538 | 40.8779 | 50.5607 |
| 2.5 | 62.8263 | 54.1779 | 55.7124 | 62.0331 | 53.8159 | 52.1987 | 51.933 | 60.1502 | 57.7744 |
| 5 | 86.8541 | 90.3885 | 78.0023 | 80.0886 | 71.2711 | 87.578 | 60.6122 | 74.6708 | 75.3331 |

TABLE 19

| Concentration of | Inhibition rate on EV71 virus (%) | | | | | | | | |
|---|---|---|---|---|---|---|---|---|---|
| polypeptide (μM) | RSQ | | | SAQ | | | AQ | | |
| 0.15625 | 1.47093 | 6.58837 | 2.82634 | 3.54678 | 2.72622 | 6.81941 | 9.88833 | 7.50558 | 5.9715 |
| 0.3125 | 11.34 | 21.2746 | 16.4228 | 8.96419 | 7.90219 | 11.0435 | 15.9607 | 11.702 | 12.6022 |
| 0.625 | 18.633 | 25.8298 | 26.1918 | 14.4055 | 11.5364 | 18.402 | 26.3573 | 23.7158 | 19.5264 |
| 1.25 | 40.5468 | 36.4575 | 35.2676 | 27.4779 | 24.0778 | 33.4848 | 33.3847 | 31.7366 | 38.7678 |
| 2.5 | 53.4848 | 40.7778 | 47.2122 | 47.0158 | 47.2468 | 36.2572 | 58.9642 | 46.161 | 41.3092 |
| 5 | 58.6022 | 68.9642 | 62.4952 | 75.1367 | 61.8367 | 63.2191 | 51.0435 | 51.8675 | 69.1952 |

TABLE 20

| Concentration of | Inhibition rate on EV71 virus (%) | | | | | | | | |
|---|---|---|---|---|---|---|---|---|---|
| polypeptide (μM) | IQ | | | GQ | | | NTQ | | |
| 0.15625 | 12.0331 | 13.5849 | 7.36773 | 7.21948 | 10.3851 | 5.4678 | 2.6541502 | 4.74383 | 5.74339 |
| 0.3125 | 18.7401 | 22.4297 | 22.9573 | 14.8883 | 12.0678 | 14.91259 | 14.830508 | 10.2542 | 13.3898 |
| 0.625 | 26.0539 | 31.1744 | 34.8606 | 28.8333 | 30.5125 | 26.48479 | 20.841686 | 30.339 | 24.339 |
| 1.25 | 41.6022 | 42.578 | 47.4848 | 43.1263 | 40.6161 | 31.07124 | 39.174089 | 47.6271 | 50.8202 |
| 2.5 | 55.7952 | 50.9781 | 60.5503 | 51.8367 | 58.9472 | 57.2507 | 45.423729 | 51.9492 | 59.661 |
| 5 | 73.2884 | 68.7332 | 83.2884 | 71.5094 | 77.9438 | 84.7439 | 68.271186 | 52.5424 | 61.6949 |

TABLE 21

| Concentration of | Inhibition rate on EV71 virus (%) | | | | | | | | |
|---|---|---|---|---|---|---|---|---|---|
| polypeptide (μM) | TQ | | | IEQ | | | TAT | | |
| 0.15625 | 3.31377 | 5.35593 | 7.23007 | 11.724 | 8.44024 | 7.62688 | 1.38042 | 5.23131 | 2.29427 |
| 0.3125 | 12.3437 | 24.3588 | 27.3869 | 18.8643 | 15.5487 | 10.6223 | 2.46255 | 5.24217 | 6.28153 |
| 0.625 | 33.3736 | 28.4011 | 30.3085 | 20.7161 | 25.7224 | 19.5166 | 8.37385 | 2.33994 | 7.23439 |
| 1.25 | 35.3209 | 40.3898 | 46.2471 | 33.6936 | 35.484 | 28.4286 | 8.36531 | 3.23706 | 6.20318 |
| 2.5 | 59.3508 | 67.3559 | 55.2235 | 45.6995 | 48.4526 | 57.4377 | 7.37254 | 4.24089 | 8.21274 |
| 5 | 71.3029 | 65.3559 | 69.2301 | 69.4739 | 61.3527 | 68.272 | 12.2608 | 8.20447 | 19.1522 |

### Example 14: Detection of Antiviral Activity of Polypeptide RQ Modifier

### 1. Experimental Materials

Polypeptides RQ-PA (shown in SEQ ID NO.16), RQ-PEG4-PA (shown in SEQ ID NO.17) and RQ-CHOL (shown in SEQ ID NO.18). The sequences were all commercially synthesized. CCK-8 reagent (MCE) was purchased from Promoter Company.

### 2. Experimental Process

(1) RD cells were plated in a 96-well plate at 100 μL per well.

(2) When the cells grew to a confluence of 70%-80%, the DMEM medium containing 10% serum was replaced with a DMEM medium containing 2% serum.

(3) The polypeptide drug was gradiently diluted with the DMEM containing 2% FBS into concentrations of 0.15625 μM, 0.3125 μM, 0.625 μM, 1.25 μM, 2.5 μM, and 5 μM, and then added to a new 96-well plate at 100 μL per well, with 3 replicate wells for each concentration. (4) The diluted virus was added to the above wells at 100 μL per well. The wells with no drug and no virus added and the wells with no drug but virus added were set as controls respectively, with a final concentration of virus of 0.1 MOI.

(4) The mixture was transferred into the 96-well plate plated with cells for another 24 h of culture, and the inhibitory activity of the polypeptide on the virus was determined by a CCK8 kit.

(5) The inhibition rates of different concentrations of the polypeptides on virus infection were calculated according to a calculation formula of: inhibition rate of polypeptide=(well with drug-well with virus)×100%/(well with no drug-well with virus).

As shown in Table 22 and FIG. 14, the determination of the inhibitory activity of the polypeptides on the virus by a CCK8 method indicates that RQ-PA had an IC50 of 3.58 μM, RQ-PEG4-PA had an IC50 of 3.47 μM, and RQ-CHOL had an IC50 of 4.25 p.M.

TABLE 22

| Concentration of | Inhibition rate on EV71 virus (%) | | | | | | | | |
|---|---|---|---|---|---|---|---|---|---|
| polypeptide (μM) | RQ-PA | | | RQ-PEG4-PA | | | RQ-CHOL | | |
| 0.15625 | 1.01454 | 2.28411 | 5.27651 | 7.27824 | 4.14494 | 8.12615 | 5.14141 | 6.97113 | 3.01327 |
| 0.3125 | 7.00885 | 10.3563 | 13.2967 | 11.2937 | 10.1514 | 13.1404 | 10.1389 | 15.972 | 11.0215 |
| 0.625 | 14.0139 | 21.402 | 20.3253 | 25.328 | 21.1587 | 27.1395 | 21.154 | 32.986 | 27.0328 |
| 1.25 | 26.9703 | 29.3073 | 30.2687 | 30.2434 | 35.1393 | 34.127 | 30.1246 | 38.9729 | 35.9495 |
| 2.5 | 35.0322 | 41.4947 | 50.486 | 44.43 | 40.1798 | 45.1788 | 34.1616 | 42.9746 | 41.0404 |
| 5 | 53.0512 | 57.3238 | 59.3155 | 62.2879 | 53.136 | 52.1554 | 49.1465 | 51.9169 | 52.0145 |

### Example 15: Determination of Efficiency of RQ-DRI in Inhibiting EV71 in RD Cells

### 1. Experimental Materials

RD cells; DMEM medium (Thermo) and serum (Gibco) were purchased from Invitrogen Co., Ltd; Total RNA extraction kit (Omega) and One step qRT-PCR kit (Takara) were purchased from U-MeBiotech Co., Ltd. The water used in the processes of RNA extraction and qRT-PCR was DEPC water, and the entire experiment was performed in an RNase-free environment.

Polypeptide RQ-DRI was synthesized by Nanjing GenScript Co., Ltd with a sequence shown in SEQ ID NO.19.

### 2. Experimental Process

(1) Different cells were plated in a 24-well plate.

(2) When the cells grew to a confluence of 70%-80%, the DMEM medium containing 10% serum was replaced with a DMEM medium containing 2% serum (the DMEM medium containing 2% serum was added in each well at an amount of 0.5 mL), and 5 μL of EV71 virus of 1×10⁶ PFU/mL was added to each well.

(3) After 1 h, different polypeptides (RQ or control TAT) with final concentrations of 0.3125 μM, 0.625 μM, 1.25 μM, 2.5 μM and 5 μM were added respectively. The group without polypeptide added was used as a control.

(4) The samples were collected 24 h after the infection of EV71 virus, and RNA was extracted with the total RNA extraction kit.

(5) The supernatant was discarded, then 350 μL of TRK lysate was added to each well, and the plate was put on a shaker for 5 min.

(6) 350 μL of 70% ethanol (DEPC) was added to each well, and then the plate was put on a shaker for 5 min.

(7) The solution in each well was transferred to a RNA extraction column for 1 min of centrifugation at 12000 g.

(8) The solution obtained in the recovery tube was reloaded on the column again for 1 min of centrifugation at 12000 g.

(9) RNA washing buffer 1 was added in the column for 30 s of centrifugation at 12000 g.

(10) RNA washing buffer 2 was added in the column for 1 min of centrifugation at 12000 g.

(11) Step (10) was repeated.

(12) The column alone was centrifuged at 12000 g for 2 min to completely remove the residual RNA washing buffer.

(13) 50 μL of DEPC water was added to the column for 2 min of centrifugation at 12000 g.

(14) 2 μL of RNA sample was subjected to a fluorescence quantitative experiment using the one step qRT-PCR kit.

The results are shown in FIG. 15 and Table 23, which indicate that the polypeptide RQ-DRI had an IC50 of 2.05 M in RD cells.

TABLE 23

| | |
|---|---|
| Concentration of | |
| RQ-DRI (μM) | Percentage of viral RNA in RD cells (%) |

| | | | |
|---|---|---|---|
| 0 | 68.13518 | 128.4527 | 103.4121 |
| 0.3125 | 90.20158 | 150.7832 | 85.03728 |
| 0.625 | 113.2834 | 92.13482 | 108.0728 |
| 1.25 | 93.12259 | 79.48209 | 58.28617 |
| 2.5 | 24.39655 | 38.77366 | 23.45334 |
| 5 | 21.2764 | 19.04182 | 17.18671 |

### Example 16: Toxicity Assay of B-RQ in RD Cells

### 1. Experimental Materials

CCK-8 reagent (MCE) was purchased from Promoter Company. Polypeptide B-RQ was synthesized by Nanjing GenScript Co., Ltd with a sequence shown in SEQ ID NO.20.

### 2. Experimental Process

(1) RD cells were plated in a 96-well plate at 100 μL per well.

(2) When the cells grew to a confluence of 70%-80%, the DMEM medium containing 10% serum was replaced with a DMEM medium containing 2% serum, and B-RQ was added so that the final concentrations in the wells were 0.46 μM, 2.34 μM, 4.68 μM, 9.37 μM, 18.75 μM, 37.5 μM, 75 μM, and 150 μM.

(3) The samples were collected 24 h after the addition of the polypeptide, and then 10 μL of live cell detection reagent CCK-8 was added to each well and mixed well.

(4) The plate was placed at 37° C. for 2 h.

(5) The absorbance value at OD450 was detected by a microplate reader.

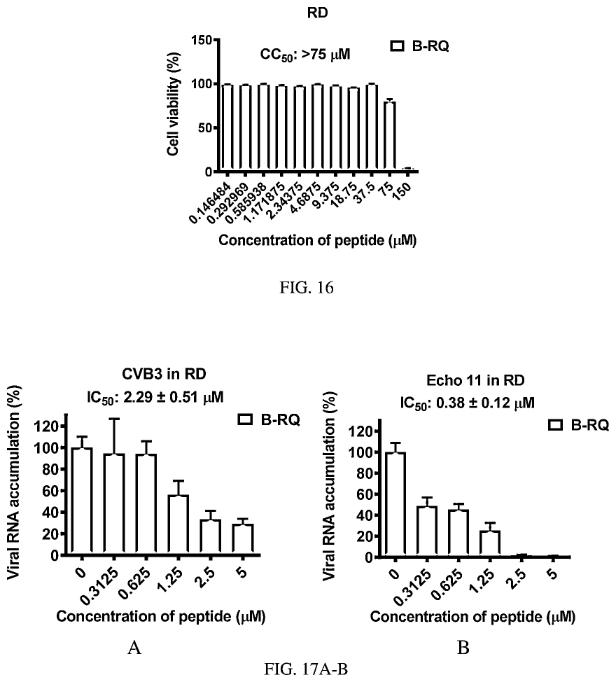

The results are shown in FIG. 16 and Table 24, which indicate that B-RQ had a CC50 of >75 μM taking the cell viability of untreated cells as 100%,.

TABLE 24

| | |
|---|---|
| Concentration of B-RQ | |
| polypeptide (μM) | Cell viability (%) |

| | | | |
|---|---|---|---|
| 0.46875 | 99.01384 | 99.46899 | 98.27423 |
| 2.34375 | 99.12763 | 97.93287 | 97.19325 |
| 4.6875 | 98.67248 | 100.7775 | 96.96567 |
| 9.375 | 99.29831 | 96.62431 | 96.16916 |
| 18.75 | 97.81908 | 95.54333 | 97.36393 |
| 37.5 | 99.35521 | 99.63967 | 99.24142 |
| 75 | 95.99848 | 95.65712 | 99.29831 |
| 150 | 95.54333 | 95.94159 | 95.31576 |

### Example 17: Determination of Efficiency of B-RQ in Inhibiting CVB3 and Echo 11 in RD Cells

### 1. Experimental Materials

RD cells; DMEM medium (Thermo) and serum (Gibco) were purchased from Invitrogen Co., Ltd; Total RNA extraction kit (Omega) and One step qRT-PCR kit (Takara) were purchased from U-MeBiotech Co., Ltd. The water used in the processes of RNA extraction and qRT-PCR was DEPC water, and the entire experiment was performed in an RNase-free environment.

Polypeptide B-RQ was synthesized by Nanjing GenScript Co., Ltd with a sequence shown in SEQ ID NO.20.

### 2. Experimental Process

(1) Different cells were plated in a 24-well plate.

(2) When the cells grew to a confluence of 70%-80%, the DMEM medium containing 10% serum was replaced with a DMEM medium containing 2% serum (the DMEM medium containing 2% serum was added in each well at an amount of 0.5 mL), and 5 μL of EV71 virus of 1×10⁶ PFU/mL was added to each well.

(3) After 1 h, the polypeptides with final concentrations of 0.3125 μM, 0.625 μM, 1.25 μM, 2.5 μM and 5 μM were added respectively. The group without polypeptide added was used as a control.

(4) The samples were collected 24 h after the infection of EV71 virus, and RNA was extracted with the total RNA extraction kit.

(5) The supernatant was discarded, then 350 μL of TRK lysate was added to each well, and the plate was put on a shaker for 5 min.

(6) 350 μL of 70% ethanol (DEPC) was added to each well, and then the plate was put on a shaker for 5 min.

(7) The solution in each well was transferred to a RNA extraction column for 1 min of centrifugation at 12000 g.

(8) The solution obtained in the recovery tube was reloaded on the column again for 1 min of centrifugation at 12000 g.

(9) RNA washing buffer 1 was added in the column for 30 s of centrifugation at 12000 g.

(10) RNA washing buffer 2 was added in the column for 1 min of centrifugation at 12000 g.

(11) Step (10) was repeated.

(12) The column alone was centrifuged at 12000 g for 2 min to completely remove the residual RNA washing buffer.

(13) 50 μL of DEPC water was added to the column for 2 min of centrifugation at 12000 g.

(14) 2 μL of RNA sample was subjected to a fluorescence quantitative experiment using the one step qRT-PCR kit.

The results are shown in FIG. 17 and Tables 25-26, which indicate that the polypeptide B-RQ had an IC50 of 2.29 μM for inhibiting CVB3 in RD cells (FIG. 17A), and an IC50 of 0.38 μM for inhibiting Echo 11 in RD cells (FIG. 17B).

TABLE 25

| Concentration of B-RQ (μM) | Percentage of CVB3 viral RNA (%) | | |
|---|---|---|---|
| 0 | 110.1778 | 109.7637 | 80.05847 |
| 0.3125 | ND | 126.7007 | 62.25837 |
| 0.625 | 116.425 | 88.76191 | 77.23717 |
| 1.25 | 80.22625 | 52.01695 | 36.74467 |
| 2.5 | 26.8204 | 49.13786 | 24.49975 |
| 5 | 22.86381 | 37.99408 | 26.97489 |

ND, not detected

TABLE 26

| Concentration of | | | | | | |
|---|---|---|---|---|---|---|
| B-RQ (μM) | Percentage of Echo 11 viral RNA (%) | | | | | |
| 0 | 90.48197 | 75.34791 | 134.1701 | 105.4064 | 82.09798 | 112.4956 |
| 0.3125 | 78.63341 | 46.42134 | 35.11162 | 43.24912 | 24.63388 | 64.67318 |
| 0.625 | 50.9076 | 51.05015 | 20.07373 | 51.16597 | 51.93671 | 48.38548 |
| 1.25 | 47.75375 | 35.28912 | 14.8342 | 3.086194 | 12.76211 | 39.50013 |
| 2.5 | 0.685338 | 0.532339 | 0.784078 | 4.1758 | 1.177338 | 3.252157 |
| 5 | 0.616862 | 1.284713 | 0.869355 | 1.744232 | 0.571171 | 1.849904 |

The above are only preferred embodiments of the present invention. It should be noted that for those of ordinary skill in the art, multiple improvements and modifications can be made without departing from the principle of the present invention, and these improvements and modifications should be considered as the protection scope of the present invention.

1. A polypeptide used as a broad-spectrum anti-enterovirus inhibitor, having a sequence selected from the group consisting of:
I.
(X1)E(X2)(X3)(X4)R(X5)(X6)(X7)(X8)(X9)(X10)(X11)
EALFQ
wherein:
X1 is selected from the group consisting of arginine (R), asparagine (N) and lysine (K);
X2 is selected from the group consisting of tyrosine (Y) and arginine (R);
X3 is selected from the group consisting of serine(S), asparagine (N) and arginine (R);
X4 is selected from the group consisting of asparagine (N), arginine (R), threonine (T) and histidine (H);
X5 is selected from the group consisting of serine(S), asparagine (N) and histidine (H);
X6 is selected from the group consisting of alanine (A), asparagine (N) and serine(S);
X7 is selected from the group consisting of isoleucine (I), threonine (T) and valine (V);
X8 is selected from the group consisting of glycine (G) and glutamine (Q);
X9 is selected from the group consisting of asparagine (N), aspartic acid (D) and alanine (A);
X10 is selected from the group consisting of threonine (T), cysteine (C) and lysine (K);
X11 is selected from the group consisting of isoleucine (I) and leucine (L);
II. a sequence with deletion, addition or substitution of at least one amino acid compared to the sequence in I;
III. a sequence that has at least 50% homology with the amino acid sequence in I or II and inhibits enterovirus activity; and
IV. a complementary sequence to the sequence in I or II or III.

2. The polypeptide according to claim 1, having a sequence shown in SEQ ID NO.1 or SEQ ID NO.24, or a D-configuration polypeptide thereof.

3. The polypeptide according to claim 1, comprising a sequence shown in SEQ ID NO.1 or SEQ ID NO.24.

4. The polypeptide according to claim 2, wherein the polypeptide has a sequence shown in SEQ ID NO.2 or SEQ ID NO.20.

5. The polypeptide according to claim 1, having a sequence with addition of 1-5 amino acids to the N-terminal of the polypeptide with the sequence shown in SEQ ID NO.21, with deletion of 1-13 amino acids from the N-terminal of the polypeptide with the sequence shown in SEQ ID NO.21, or with modification to the C-terminal of the polypeptide with the sequence shown in SEQ ID NO.21, or a D configuration polypeptide thereof.

6. The polypeptide according to claim 5, further comprising a cell-penetrating peptide.

7. The polypeptide according to claim 6, having a sequence shown in any one of SEQ ID NOs.3-20.

8. A method for inhibiting an enterovirus, comprising administering the polypeptide according to claim 1 to a subject in need thereof.

9. A method for treating or preventing enterovirus infection, comprising administering the polypeptide according to claim 1 to a subject in need thereof.

10. The method according to claim 8, wherein the enterovirus is selected from the group consisting of human enterovirus (EV), coxsackie A virus (CVA), coxsackie B virus (CVB), echovirus, rhinovirus and poliovirus.

11. The method according to claim 9, wherein the enterovirus infection causes a disease selected from the group consisting of hand-foot-mouth disease, myocarditis, herpetic angina, aseptic meningitis, encephalitis, and viral cold.

12. A method for preventing and/or treating a viral disease, comprising administering a preparation inhibiting multimerization of enterovirus protein 2C as a target to a subject in need thereof.

13. The method according to claim 9, wherein the enterovirus is selected from the group consisting of human enterovirus (EV), coxsackie A virus (CVA), coxsackie B virus (CVB), echovirus, rhinovirus and poliovirus.


SEQUENCE LISTING (24 sequences)

SEQ ID NO: 1 - protein, 18 aa, Artificial Sequence
  Core Sequence
REYNNRSAIGNTIEALFQ

SEQ ID NO: 2 - protein, 32 aa, Artificial Sequence
  Polypeptide RQ
YGRKKRRQRRRGSGREYNNRSAIGNTIEALFQ

SEQ ID NO: 3 - protein, 34 aa, Artificial Sequence
  Polypeptide LQ
YGRKKRRQRRRGSGLIREYNNRSAIGNTIEALFQ

SEQ ID NO: 4 - protein, 36 aa, Artificial Sequence
  Polypeptide SQ
YGRKKRRQRRRGSGSELIREYNNRSAIGNTIEALFQ

SEQ ID NO: 5 - protein, 31 aa, Artificial Sequence
  Polypeptide EQ
YGRKKRRQRRRGSGEYNNRSAIGNTIEALFQ

SEQ ID NO: 6 - protein, 30 aa, Artificial Sequence
  Polypeptide YQ
YGRKKRRQRRRGSGYNNRSAIGNTIEALFQ

SEQ ID NO: 7 - protein, 29 aa, Artificial Sequence
  Polypeptide NQ
YGRKKRRQRRRGSGNNRSAIGNTIEALFQ

SEQ ID NO: 8 - protein, 27 aa, Artificial Sequence
  Polypeptide RSQ
YGRKKRRQRRRGSGRSAIGNTIEALFQ

SEQ ID NO: 9 - protein, 26 aa, Artificial Sequence
  Polypeptide SAQ
YGRKKRRQRRRGSGSAIGNTIEALFQ

SEQ ID NO: 10 - protein, 25 aa, Artificial Sequence
  Polypeptide AQ
YGRKKRRQRRRGSGAIGNTIEALFQ

SEQ ID NO: 11 - protein, 24 aa, Artificial Sequence
  Polypeptide IQ
YGRKKRRQRRRGSGIGNTIEALFQ

SEQ ID NO: 12 - protein, 23 aa, Artificial Sequence
  Polypeptide GQ
YGRKKRRQRRRGSGGNTIEALFQ

SEQ ID NO: 13 - protein, 22 aa, Artificial Sequence
  Polypeptide NTQ
YGRKKRRQRRRGSGNTIEALFQ

SEQ ID NO: 14 - protein, 21 aa, Artificial Sequence
  Polypeptide TQ
YGRKKRRQRRRGSGTIEALFQ

SEQ ID NO: 15 - protein, 20 aa, Artificial Sequence
  Polypeptide IEQ
YGRKKRRQRRRGSGIEALFQ

SEQ ID NO: 16 - protein, 19 aa, Artificial Sequence
  Polypeptide RQ-PA
  UNSURE (1)..(19): Xaa 19 =beta-AK-C16
REYNNRSAIGNTIEALFQX

SEQ ID NO: 17 - protein, 19 aa, Artificial Sequence
  Polypeptide RQ-PEG4-PA
  UNSURE (1)..(19): Xaa 19 =beta-AK-PEG4-K-C16
REYNNRSAIGNTIEALFQX

SEQ ID NO: 18 - protein, 19 aa, Artificial Sequence
  Polypeptide RQ-CHOL
  UNSURE (1)..(19): Xaa 19 =beta-AK-Chol
REYNNRSAIGNTIEALFQX

SEQ ID NO: 19 - protein, 32 aa, Artificial Sequence
  Polypeptide RQ-DRI
QFLAEITNGIASRNNYERGSGRRRQRRKKRGY

SEQ ID NO: 20 - protein, 32 aa, Artificial Sequence
  Polypeptide B-RQ
YGRKKRRQRRRGSGREYNHRHSVGATLEALFQ

SEQ ID NO: 21 - protein, 18 aa, Artificial Sequence
  Core Sequence
  UNSURE (1)..(1): Xaa (1) = arginine (R) or asparagine (N) or lysine (K)
  UNSURE (3)..(3): Xaa (3) = tyrosine (Y) or arginine (R)
  UNSURE (4)..(4): Xaa (4) = serine (S) or asparagine (N) or arginine (R)
  UNSURE (5)..(5): Xaa (5) = asparagine (N) or arginine (R) or threonine (T) or histidine (H)
  UNSURE (7)..(7): Xaa (7) = serine (S) or asparagine (N) or histidine (H)
  UNSURE (8)..(8): Xaa (8) = alanine (A) or asparagine (N) or serine (S)
  UNSURE (9)..(9): Xaa (9) = isoleucine (I) or threonine (T) or valine (V)
  UNSURE (10)..(10): Xaa (10) = glycine (G) or glutamine (Q)
  UNSURE (11)..(11): Xaa (11) = asparagine (N) or aspartic acid (D) or alanine (A)
  UNSURE (12)..(12): Xaa (12) = threonine (T) or cysteine (C) or lysine (K)
  UNSURE (13)..(13): Xaa (13) = isoleucine (I) or leucine (L)
XEXXXRXXXXXXXEALFQ

SEQ ID NO: 22 - protein, 18 aa, Artificial Sequence
  Core Sequence
  UNSURE (5)..(5): Xaa (5) = asparagine (N) or arginine (R) or threonine (T) or histidine (H)
  UNSURE (7)..(7): Xaa (7) = serine (S) or asparagine (N) or histidine (H)
  UNSURE (8)..(8): Xaa (8) = alanine (A) or asparagine (N) or serine (S)
  UNSURE (9)..(9): Xaa (9) = isoleucine (I) or threonine (T) or valine (V)
  UNSURE (10)..(10): Xaa (10) = glycine (G) or glutamine (Q)
  UNSURE (11)..(11): Xaa (11) = asparagine (N) or aspartic acid (D) or alanine (A)
  UNSURE (12)..(12): Xaa (12) = threonine (T) or cysteine (C) or lysine (K)
  UNSURE (13)..(13): Xaa (13) = isoleucine (I) or leucine (L)
REYNXRXXXXXXXEALFQ

SEQ ID NO: 23 - protein, 18 aa, Artificial Sequence
  Core Sequence
  UNSURE (5)..(5): Xaa (5) = asparagine (N) or arginine (R) or threonine (T) or histidine (H)
  UNSURE (7)..(7): Xaa (7) = serine (S) or asparagine (N) or histidine (H)
  UNSURE (8)..(8): Xaa (8) = alanine (A) or asparagine (N) or serine (S)
  UNSURE (9)..(9): Xaa (9) = isoleucine (I) or threonine (T) or valine (V)
  UNSURE (11)..(11): Xaa (11) = asparagine (N) or aspartic acid (D) or alanine (A)
  UNSURE (13)..(13): Xaa (13) = isoleucine (I) or leucine (L)
REYNXRXXXGXTXEALFQ

SEQ ID NO: 24 - protein, 18 aa, Artificial Sequence
  Core Sequence
REYNHRHSVGATLEALFQ